(19) United States
(12) Patent Application Publication
(10) Pub. No.: US 2025/0295629 A1
(43) Pub. Date: Sep. 25, 2025

(54) COMPOSITIONS AND METHODS FOR TREATING CANCERS OF THE CENTRAL NERVOUS SYSTEM (CNS), INCLUDING GLIOBLASTOMA AND CHEMORESISTANT CNS TUMORS, AND RELATED COMPOSITIONS AND METHODS FOR INHIBITING AND ELIMINATING CNS CANCER STEM CELLS

(71) Applicant: Daniel Pierce RADIN, Glen Rock, NJ (US)

(72) Inventor: Daniel Pierce RADIN, Glen Rock, NJ (US)

(21) Appl. No.: 19/231,941

(22) Filed: Jun. 9, 2025

Related U.S. Application Data
(63) Continuation of application No. 18/719,246, filed on Jun. 13, 2024, now abandoned.
(60) Provisional application No. 63/421,503, filed on Nov. 1, 2022.

(51) Int. Cl. A61K 31/382; A61K 31/495; A61P 35/00

(52) U.S. Cl. CPC A61K 31/382; A61K 31/495; A61P 35/00

(57) ABSTRACT

Novel compositions and methods are provided for effective clinical management of central nervous system (CNS) cancers in mammalian subjects, including humans. The anti-CNS cancer compositions and methods employ lucanthone, alone or in combination with other anti-CNS cancer drugs or methods (including chemotherapy, such as with temozolomide, and radiation), to prevent or reduce CNS cancer, including gliomas. In certain embodiments, lucanthone compositions and methods effectively treat glioblastomas, including high grade glioblastomas. In other embodiments, lucanthone compositions and methods effectively target and control CNS cancer stem cells, for example to reduce or prevent recurrence of glioblastomas unresolved by conventional treatments.

### TECHNICAL FIELD

The invention relates to methods and compositions for treating cancers in mammalian subjects. More particularly, the invention relates to methods and compositions for treating cancers of the central nervous system (CNS), including glioblastoma, in human subjects.

### BACKGROUND OF THE INVENTION

Among cancers of the central nervous system (CNS), glioblastoma is the most common and aggressive primary brain tumor in human adults. Median survival for glioblastoma patients remains 16-20 months, even with current standard multimodal treatment employing surgical resection, radiation, temozolomide and tumor-treating fields therapies.

Many factors are speculated to contribute to glioblastoma treatment resistance, though none are well understood. Reports suggest that genotoxic chemotherapy may prompt glioma cells to initiate cytoprotective autophagy, which may contribute to treatment resistance and glioma recurrence. The concept of blocking or inhibiting specific steps in the autophagy pathway has been proposed, as a possible strategy for enhancing efficacy of both classical chemotherapies and newer immune-stimulating therapies. However, the prospect of interfering with autophagy in the clinic has heretofore been frustrated by low potency and bioavailability of candidate autophagy inhibitors, including poor ability to cross the blood-brain barrier.

In view of the low survival and poor treatment options for glioblastoma patients and patients with other chemoresistant CNS cancers, a long unmet, urgent persists in the art for new clinical management tools to treat these exceptionally lethal and treatment-refractory cancers.

### SUMMARY OF EXEMPLARY EMBODIMENTS OF THE INVENTION

The instant invention fulfills the foregoing needs and satisfies additional objects and advantages by providing novel methods and compositions for treating or preventing central nervous system (CNS) cancers in mammalian subjects, employing potent anti-CNS cancer effective lucanthone compounds, compositions and methods.

CNS cancers, such as glioblastoma, are effectively treated according to the invention by administering to a CNS cancer-presenting or at-risk subject a lucanthone compound or composition in an amount, dosage or therapeutic regimen effective to reduce one or more adverse clinical symptom(s) of a targeted CNS cancer, and/or to extend average survival among treated versus control subjects.

In certain embodiments, the lucanthone compound or composition exerts a novel, unexpectedly effective anti-CNS cancer activity, to effectively reduce an incidence or severity of the targeted CNS cancer in treated subjects, for example as evinced by a reduction in size, growth or other cancer-related activity of a treated tumor, and/or by a reduction in an incidence or severity of any one or more diagnostic side effects, such as reduced survival, attributable to the targeted CNS cancer in treated subjects. In related embodiments, lucanthone compounds, pharmaceutical compositions and treatment methods of the invention exert unprecedented anti-CNS cancer activity to effectively minimize or overcome chemotherapy resistance and/or disease recurrence of a CNS cancer in treated subjects, for example following conventional first line chemotherapy treatment. In illustrative embodiments directed at glioblastoma, lucanthone compositions and methods described herein reduce incidence and/or severity of glioblastoma chemoresistance and/or disease recurrence in patients treated with conventional glioblastoma chemotherapy, such as temozolomide. This novel efficacy can be provided by coordinate/contemporary treatment of glioblastoma patients with temozolomide and lucanthone, or by follow-on lucanthone treatment after first-line treatment with temozolomide, where in both regimens the addition of lucanthone to the therapy potently reduces incidence and/or severity of glioblastoma chemoresistance and/or disease recurrence.

In further embodiments, lucanthone compounds and compositions of the invention exhibit yet another novel and unexpected anti-CNS cancer activity, marked by potent suppression of growth and/or survival of stem cells of a targeted glioblastoma or other chemotherapy-resistant CNS cancer. In related embodiments, novel pharmaceutical compositions and treatment methods are provided employing lucanthone compounds to selectively target and impair or eliminate glioblastoma stem cells, as exemplified by effective targeting and elimination of Olig2+ glioma stem cells, associated with untreated or chemotherapy-resistant human gliomas in vivo.

In other detailed embodiments, lucanthone compositions and methods of the invention effectively treat CNS cancers, including glioblastomas, by effectively normalizing tumor vasculature in treated versus control subjects.

In further embodiments, lucanthone compositions and methods of the invention effectively treat CNS cancers, including glioblastomas, by reducing tumor hypoxia in treated versus control subjects.

Yet additional aspects of the invention include methods for treating or preventing central nervous system (CNS) cancers m mammalian subjects using a lucanthone compound or composition coordinately with a CNS cancer chemotherapeutic agent, for example a temozolomide compound or composition, in respective amounts, dosages or regimens that coordinately and complementarily (e.g., additively or synergistically) reduce one or more adverse clinical symptom(s) of the CNS cancer, and/or extend average survival among coordinately treated subjects. In related embodiments, novel pharmaceutical compositions are provided to treat or prevent CNS cancers in mammalian subjects, employing a lucanthone compound or composition co-formulated or packaged for coordinate clinical use with a secondary CNS cancer chemotherapeutic agent, such as a temozolomide compound, in respective amounts or dosages to coordinately and complementarily reduce one or more adverse clinical symptom(s) of the CNS cancer, and/or extend average survival among coordinately treated subjects.

### BRIEF DESCRIPTION OF THE DRAWINGS

FIG. 1 graphically illustrates mechanisms of lucanthone activity against glioma cells. Figure. lA Chemical structures of lucanthone and chloroquine. FIG. 1B Effects of long-term treatment of KR158 and GLUC2 cultures with 10 μM lucanthone on glioma cell proliferation. FIG. 1C Acridine orange (AO) marks lysosomes as punctae staining after 48 hours of lucanthone treatment. FIG. 1D LC3 marks autophagosome punctae levels after 48 hours treatment with lucanthone. Figure IE and 1F show effects of lucanthone on P62 and Cathepsin D levels in GLUC2 and KR158 cells, respectively. Scale bar=30 μm. Bars are mean+/−SEM. N=3-4 independent experiments. *p<0.05, ****p<0.0001, student's t-test

FIG. 2 graphically illustrates effects of lucanthone on yH2AX and Ki67 and cleaved caspase 3. GLUC2 (A) and KR158 cells (B) were treated with lucanthone or the topoisomerase 2 inhibitor etoposide for 48 hours, after which cells were stained for yH2AX. Representative photomicrographs of Ki67 and cleaved caspase 3 (CC3) in GLUC2 (C) and KR158 (D) spheroids treated with control and 10 μM lucanthone for 48 hours.

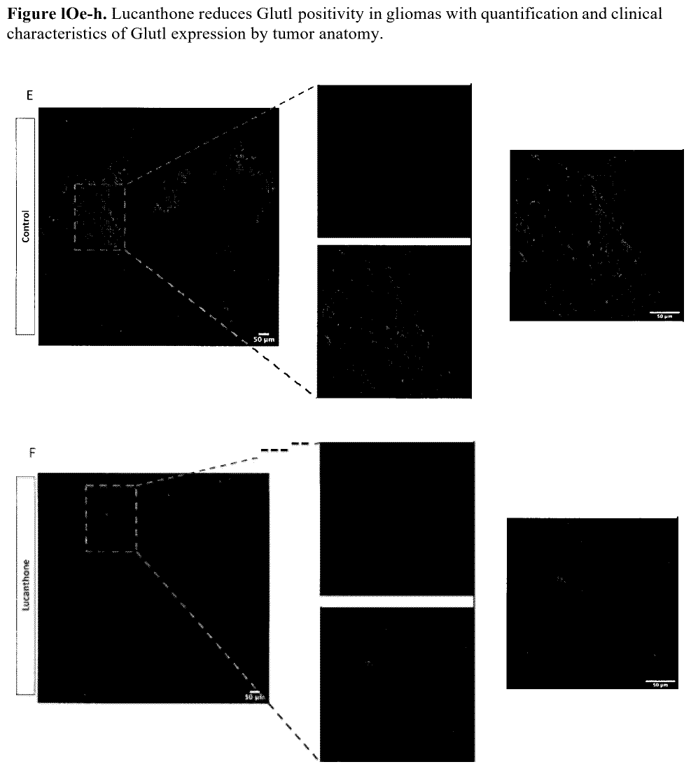

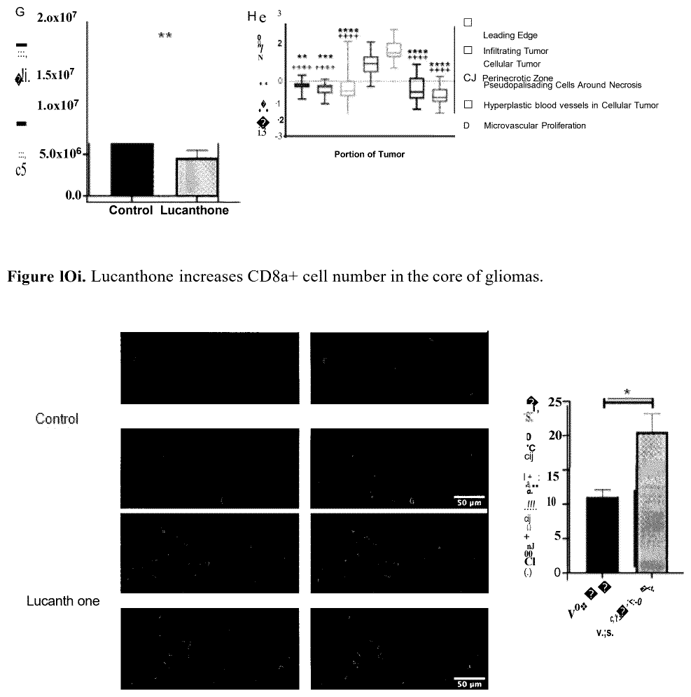

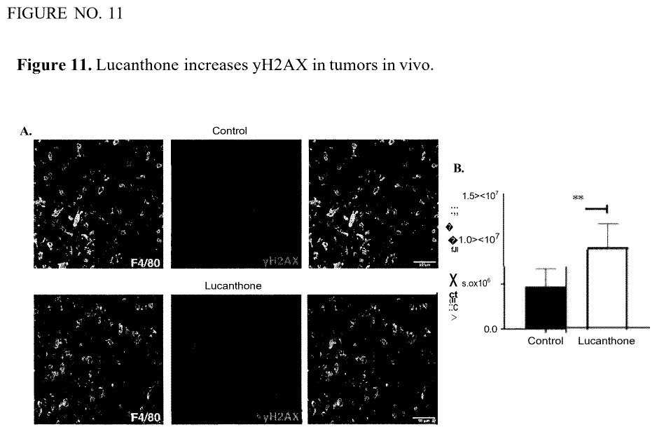

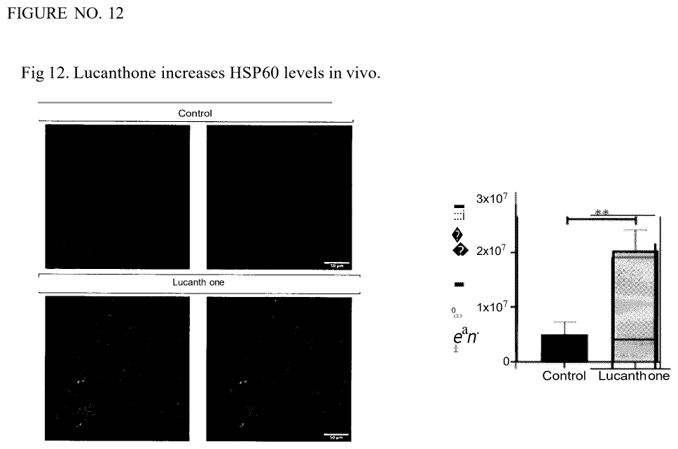

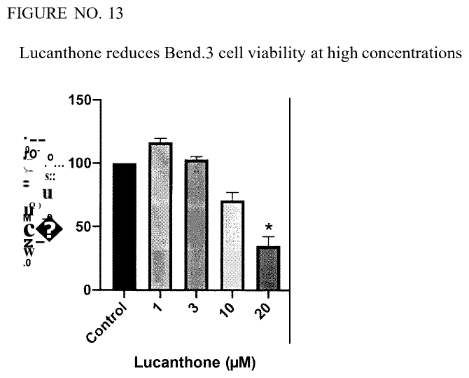

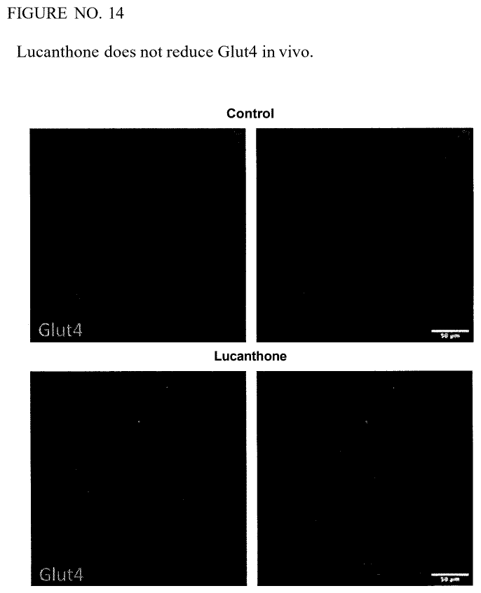

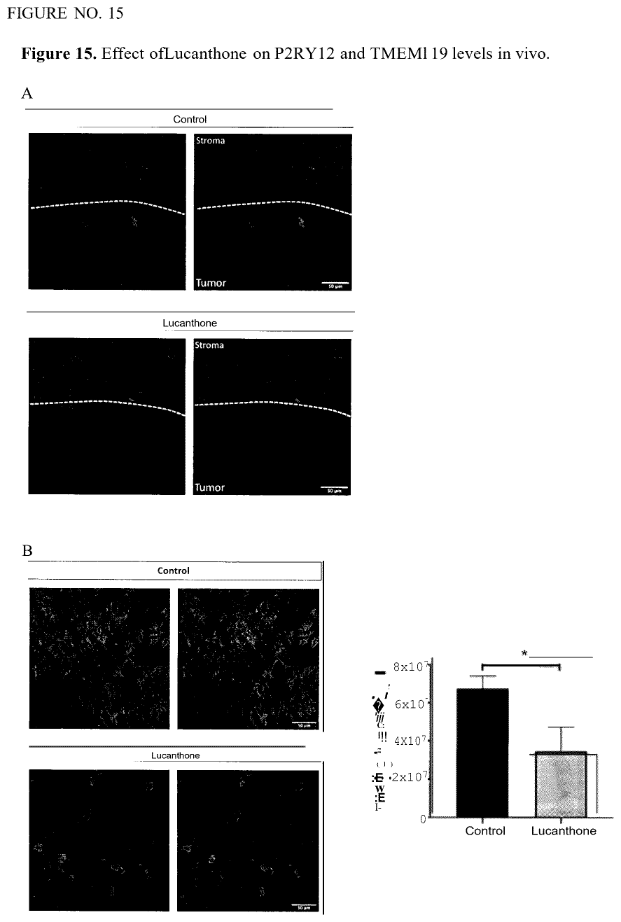

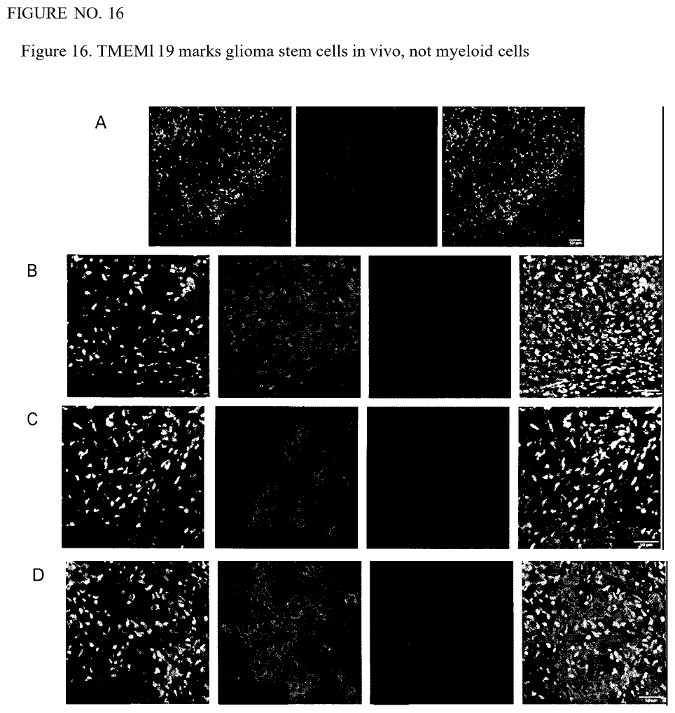

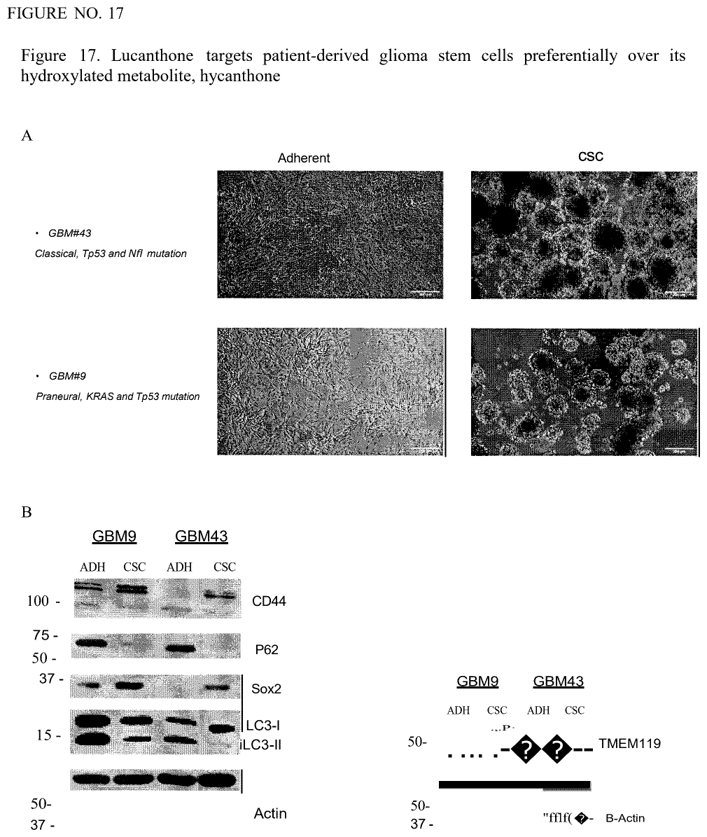

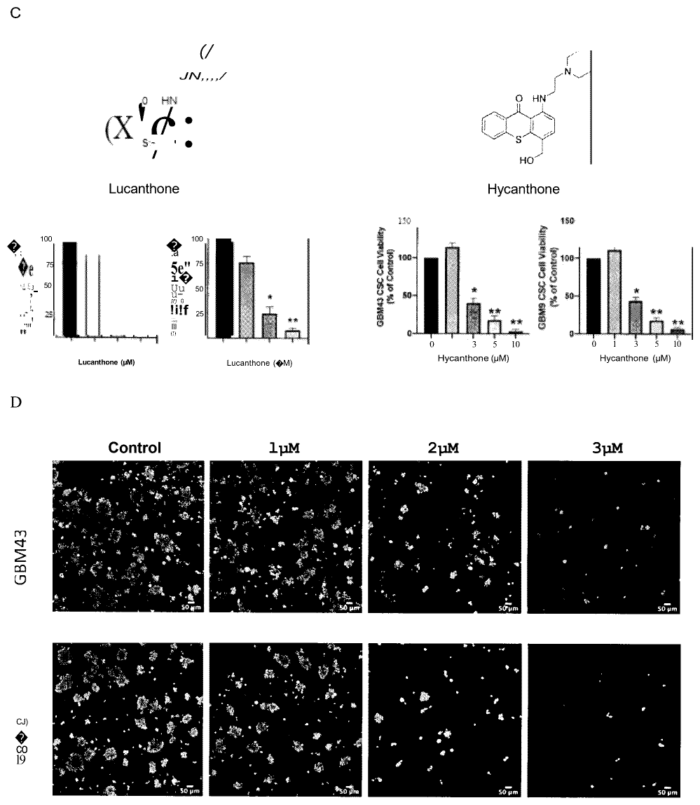

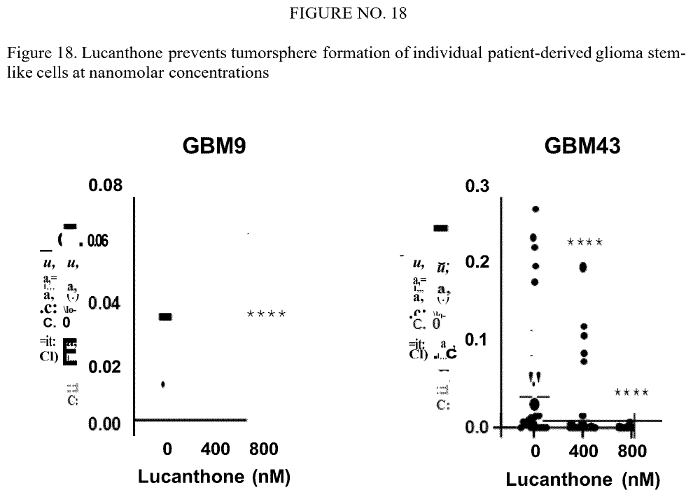

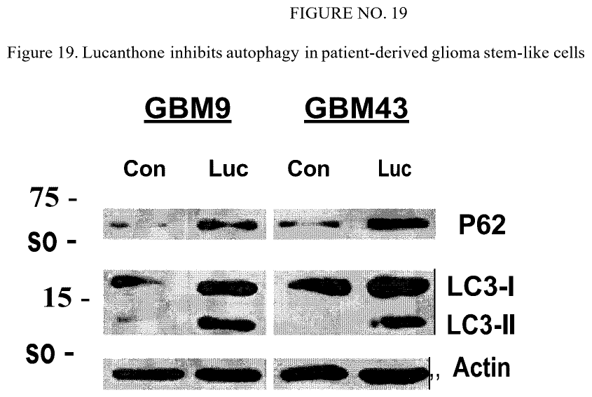

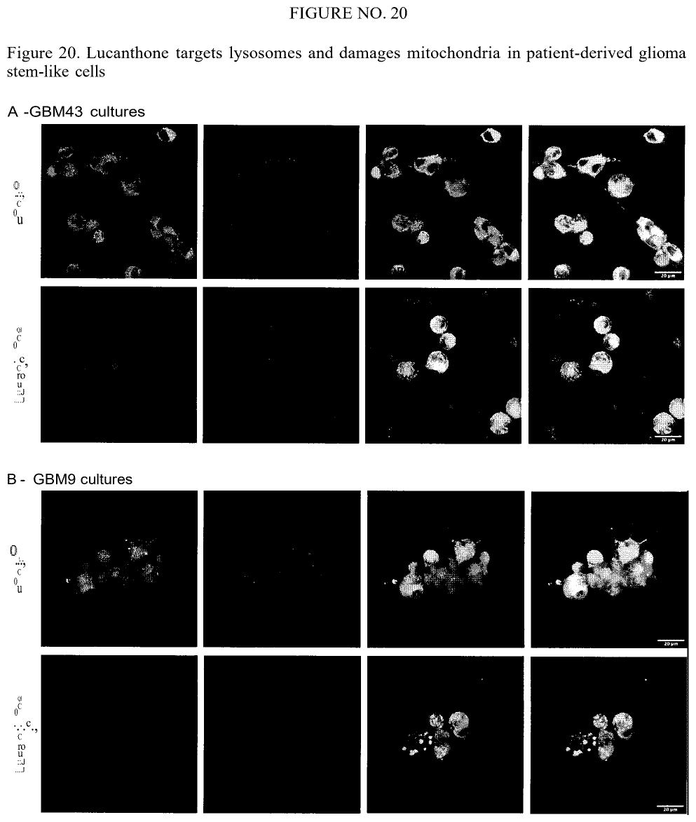

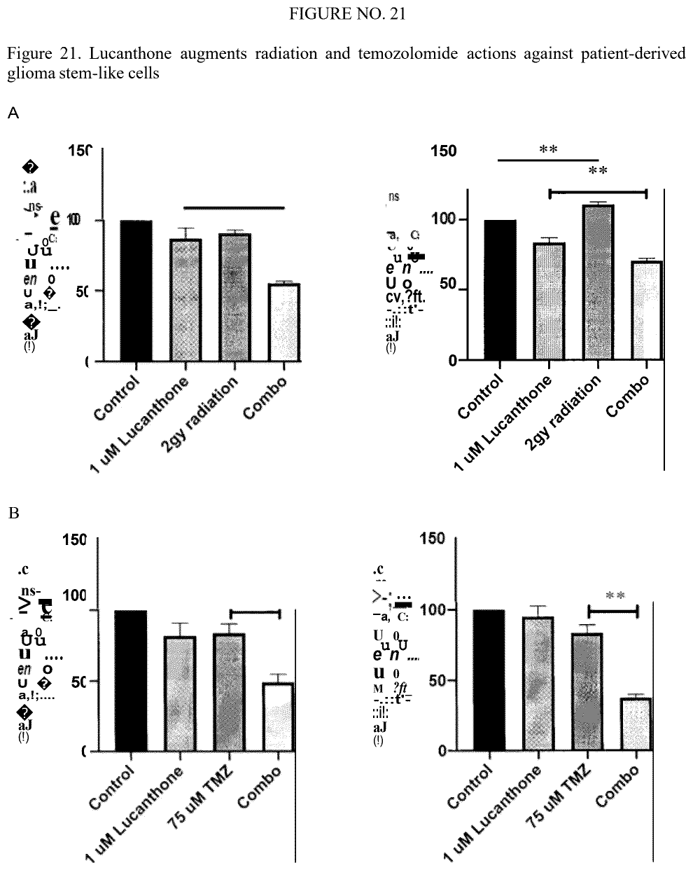

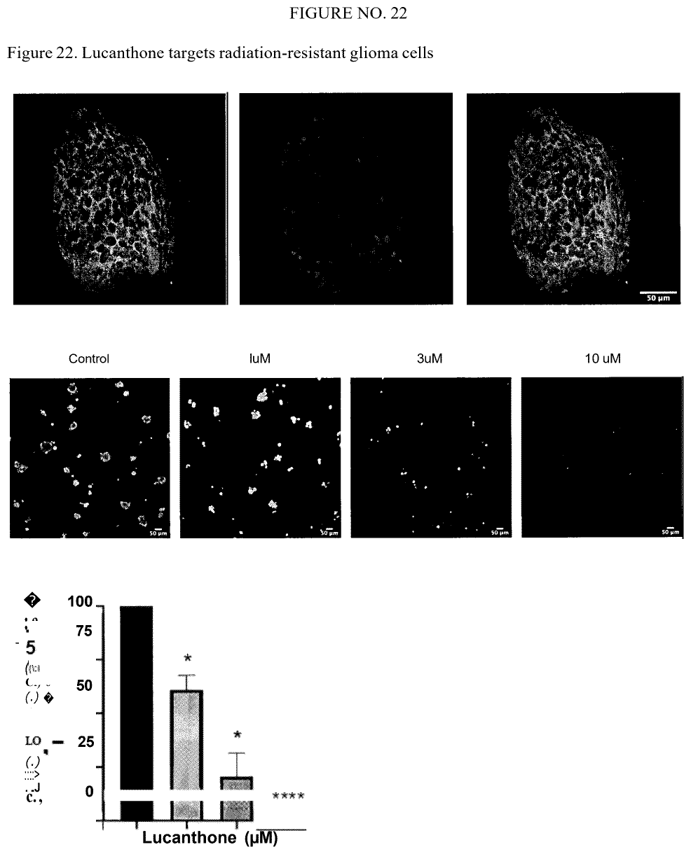

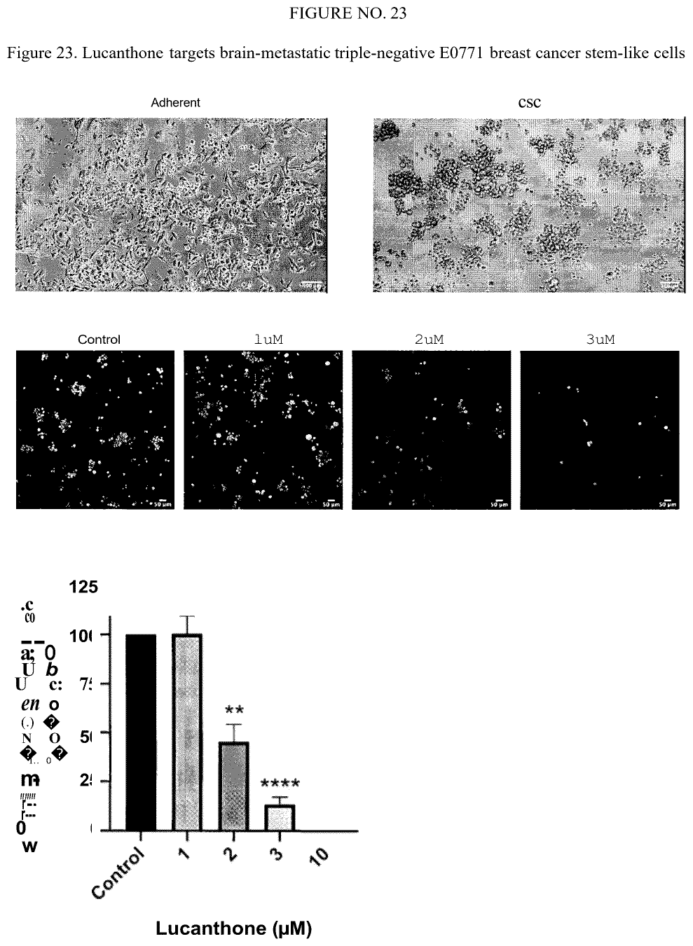

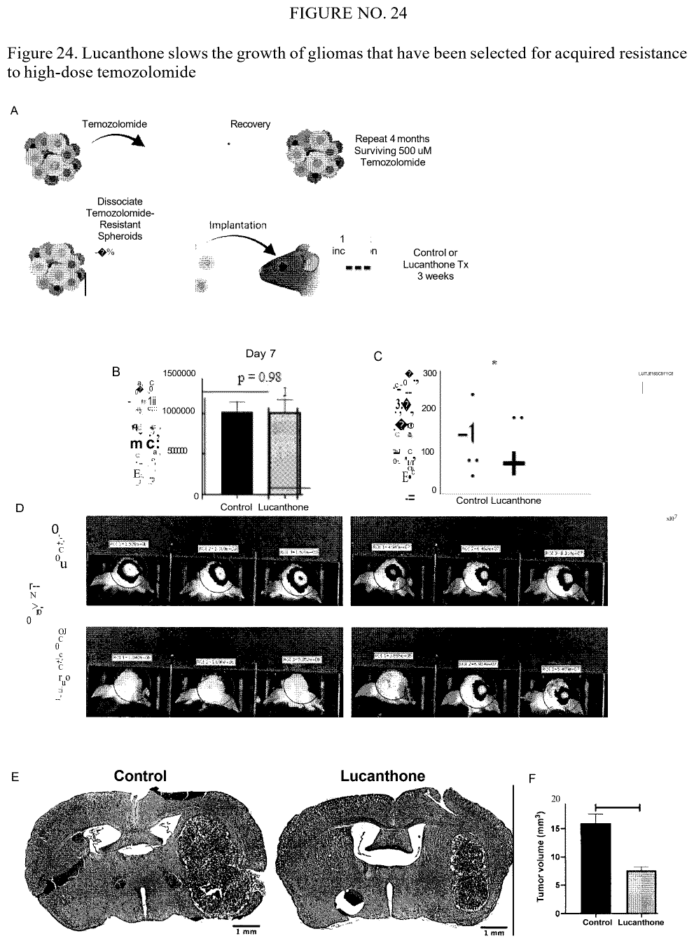

Figure[DRIJ 3 graphically illustrates individual and coordinate activities of lucanthone and temozolomide. FIG. 2A) KR158 and GLUC2 cells were treated with lucanthone for 72 hours, after which an MTT assay was performed. Bars are mean+/−SEM, N=3-7 independent experiment. ANOVA p<0.0001. *p<0.05, **p<0.01, ***p<0.001, ****p<0.0001, Dunnett's multiple comparison test to control-treated cells. FIGS. 2B and 2C KRl 58 and GLUC2 cells were treated with lucanthone, TMZ, or the combination for 4 days and then allowed to recover in drug-free medium for 3 days. The cells were PFA-fixed and stained with crystal violet. Crystal violet-stained cells were then lysed and relative absorbance was measured to approximate culture viability. Representative wells are shown in (B). FIG. 2C) Quantification of crystal-violet stained cultures. Bars are mean+/−SEM, N=3-4 independent experiments. *p<0.05, **p<0.01, Dunnett's multiple comparison test to control-treated cells. +p<0.05, King's synergy test, demonstrating significant interactions between lucanthone and TMZ in both cell lines.

FIG. 4 graphically illustrates how glioma spheroids express sternness markers (A) GLUC2 and KR1 58 spheroids were adhered to glass slides by pre-coating slides with Geltrex for an hour. After adhering for 24 hours, spheroids were PFA-fixed and stained for SOX2, nestin, Olig2, CD133 and Ki67. GLUC2 spheroids expressed SOX2, nestin, Olig2 were also positive for the proliferation marker Ki67. KR158 spheroids expressed SOX2, CD133 and nestin, in addition to Ki67. Results are representative of 3 independent experiments. B) Spheroids also express increased levels of SOX2 and Olig2 measured by western blot.

Figure[DR2J 5¹[DR3J graphically illustrates how lucanthone targets GSC and overcomes acquired resistance to temozolomide. GLUC2 and KRI 58 spheroids were mechanically dissociated, plated overnight and treated with increasing concentrations of lucanthone for 5 days. After treatment, they were stained with Calcein-AM to visualize viable cells. Figure SA Representative images of KRI 58 and GLUC2 GSC treated with increasing concentrations of lucanthone for 5 days. FIG. 5B Spheroid area distribution. ****p<0.0001, Kolmogorov-Smirnov test comparing distributions to control-treated cultures. Figure SC Spheroid number per field of view. Figure SD Viability of cultures as determined by MTT assay. Bars are mean+/−SEM, N=3-4 independent experiments. *p<0.05, **p<0.01, ***p<0.001, Dunnett's multiple comparison test to control-treated cells. FIGS. 3E and 3F LC3 intensity was measured in GLUC2 spheroid cultures treated with media or 10 μM lucanthone for 48 hours. *p<0.05, Mann-Whitney test. FIG. 50 P62 and LC3 is increased after treatment with lucanthone in GLUC2 and KR158 spheroids. SH and SI Olig2 intensity and mRNA expression was measured in GLUC2 spheroid cultures treated with media or 10 μM lucanthone for 48 hours. **p<0.01, t-test. N=3-4 independent experiments. FIG. 51 GLUC2 cells treated with 5 cycles of TMZ stained for the sternness marker CD133 and for the proliferation marker Ki67. Figure SK TMZ-resistant GLUC2 cells treated with media or 10 μM lucanthone for 5 days.

FIG. 6 graphically illustrates how Parental GLUC2 cells are more sensitive to TMZ. GLUC2 cells (parental) as well as cells that had developed resistance to TMZ were exposed to increasing concentrations of TMZ. The culture viability was examined 72 hours after treatment by MTT. Bars are mean+/−SEM, n=4 independent experiments. P<0.01, Two-way ANOVA, indicating significant effects by dose and between cell lines.

FIG. 7 ;[oR4J graphically illustrates that Patient-derived glioma cells are susceptible to lucanthone. A) GBM43 cells were treated with lucanthone and assessed for changes in acridine orange staining, B) LC3 and C) p62 levels. D) GBM43 CSCs were treated with lucanthone for 5 days and then an MTT assay was performed. E) GBM43 GSC were treated with media or lucanthone for 5 days, after which spheroids were visualized by Calcein-AM and Ethidium homodimer staining. Data are representative of 4 independent experiments. ****p<0.0001, t-test. Dotted line represents culture viability prior to any treatment.

Figure[DR5J 8 graphically illustrates how lucanthone mitigates growth of dissociated GLUC2 spheroids in vivo. FIG. 8A Treatment scheme used for the study. FIG. 8B Representative images of in vivo luminescent imaging on Days 7, 14 and 21. FIG. 8C Fold increase in luminescence from day 7 to day 21. ***p<0.001, Mann-Whitney test. FIG. 8D Tumor volume of control- and lucanthone-treated animals with representative images shown in FIG. 8E **p<0.01, Mann-Whitney test. FIG. 8F Body mass depicted as a percentage of the start of treatment on day 7. ***p<0.001, Mann-Whitney test, compared to relative body mass on day 7. Bars are mean+/−SEM, N=7-8 animals.

FIG. 9 (DR6J graphically illustrates how lucanthone reduces Olig2+ positivity in tumors in vivo. FIG. 9A Representative immunohistochemical images of Olig2 and Ki67 in tumors and surrounding stroma in saline- and lucanthone-treated mice. FIG. 9B Expression of Olig2 in different areas in human glioblastomas adapted from the Ivy Glioblastoma Atlas. ****p<0.0001 Kruskal-Wallis test, demonstrating significant differences in Olig2 expression among various tumor areas. *p<0.05, ****p<0.0001 Dunn's test, compared to infiltrating tumor. +p<0.05, ++++p<0.0001, Dunn's test, compared to cellular tumor. FIGS. 9C and 9D Olig2 expression in tumor periphery and tumor core in both treatment conditions with intensity quantifications in FIG. 9E. Two-way ANOVA p<0.05. **p<0.01, Bonferroni multiple comparison test. Bars are mean+/−SEM, N=4 animals per group.

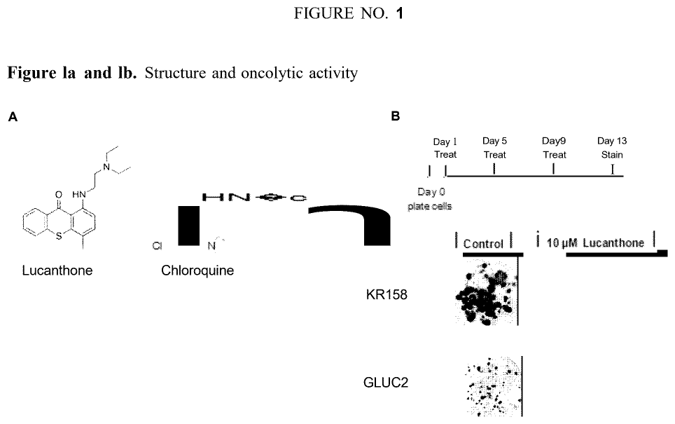

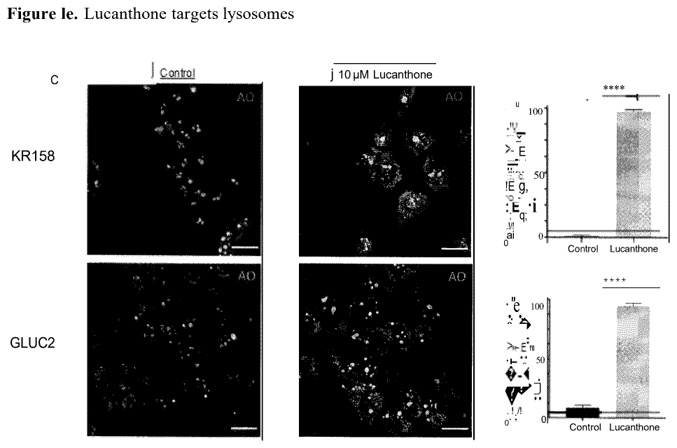

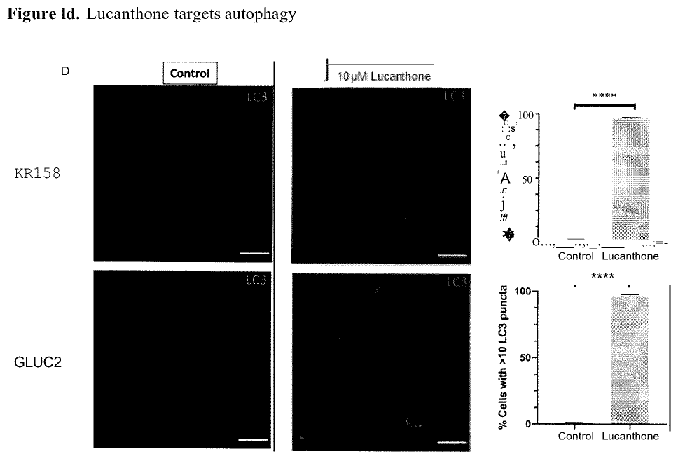

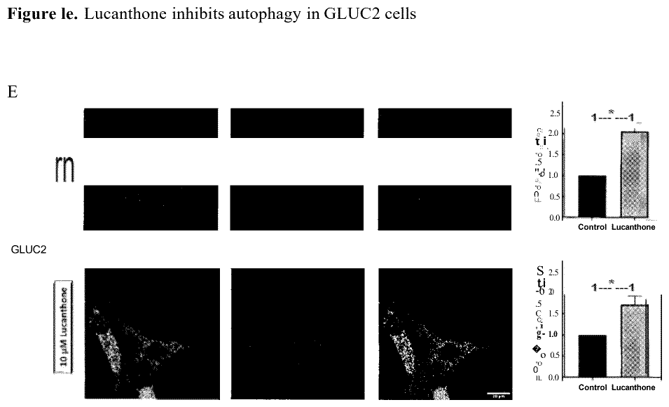

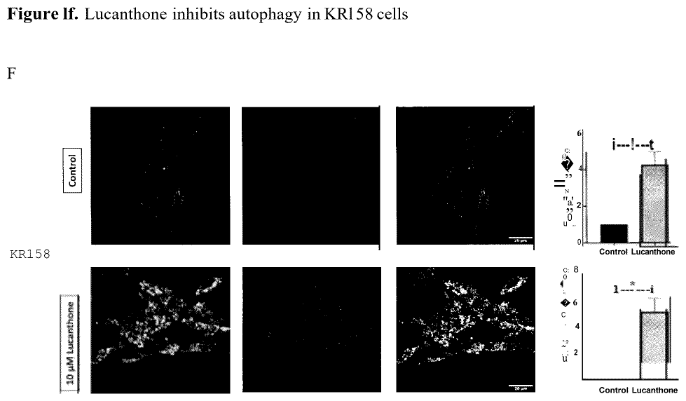

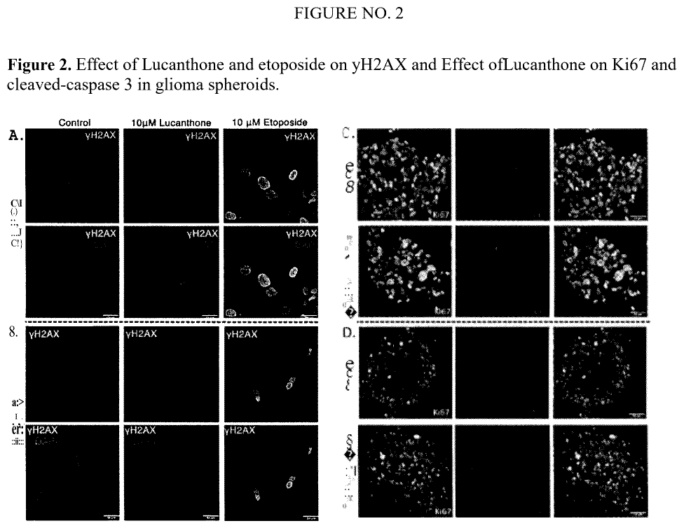

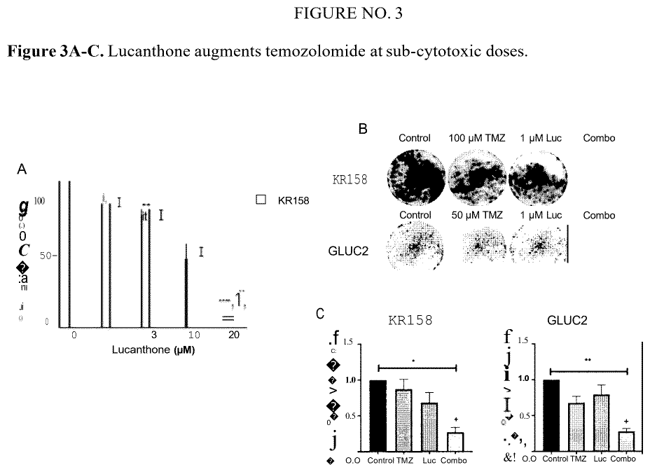

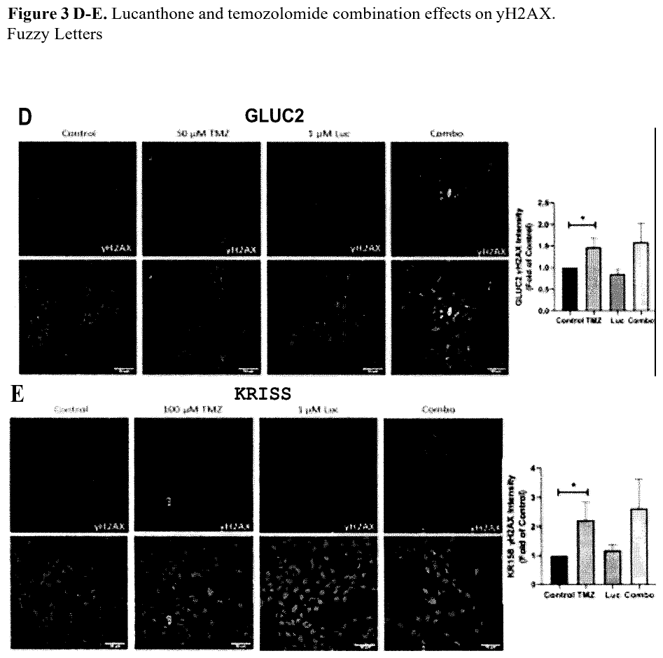

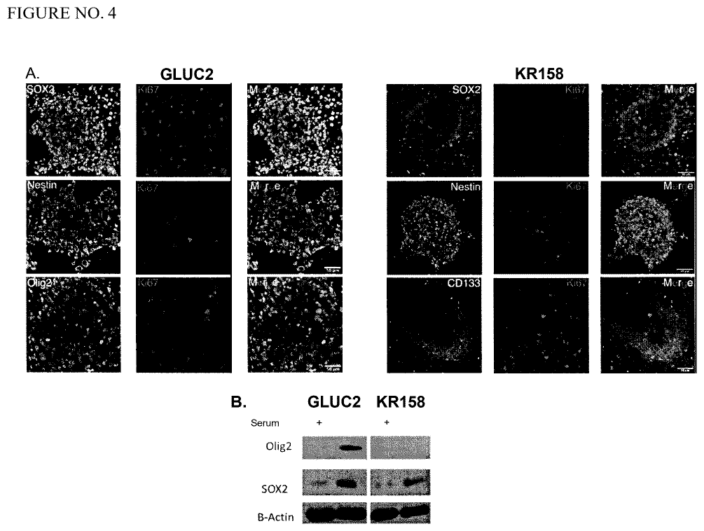

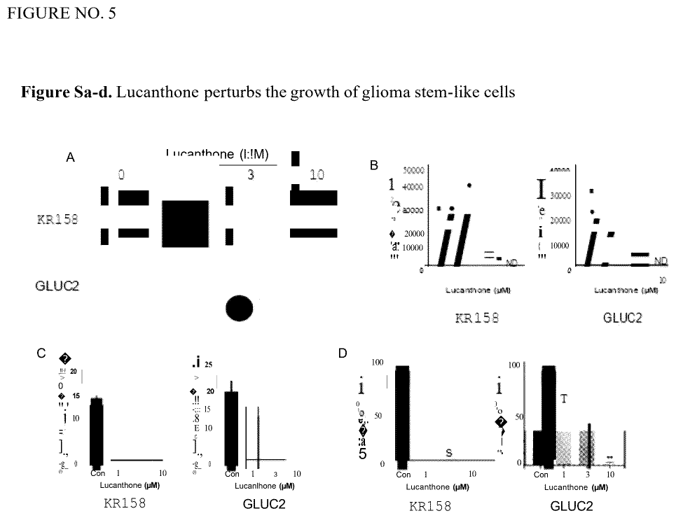

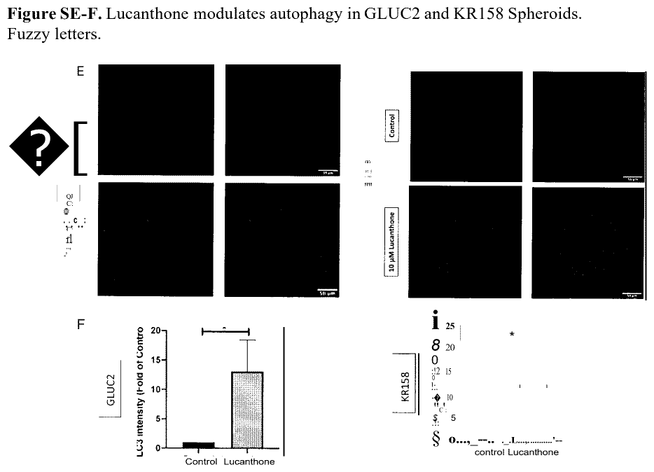

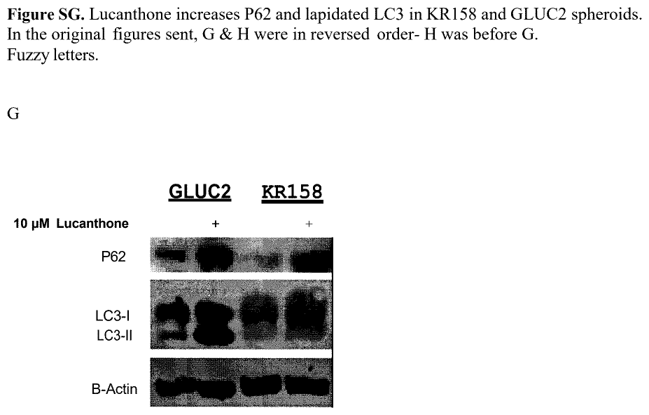

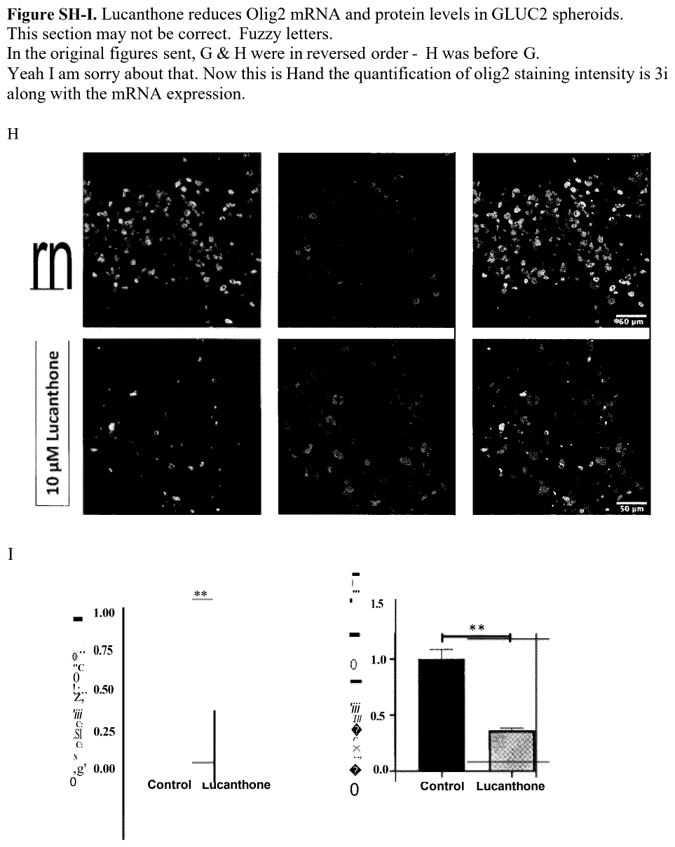

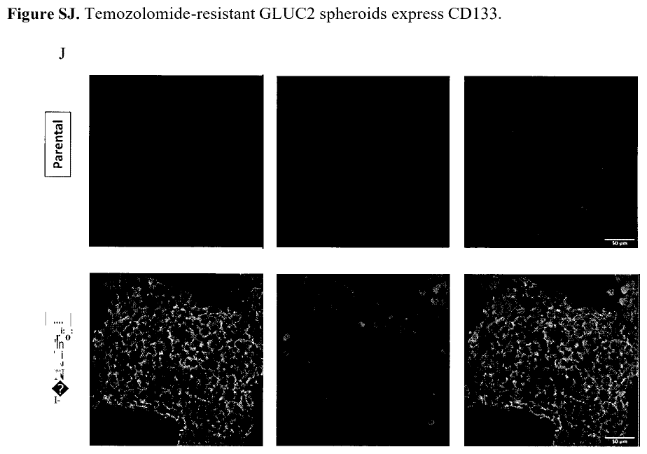

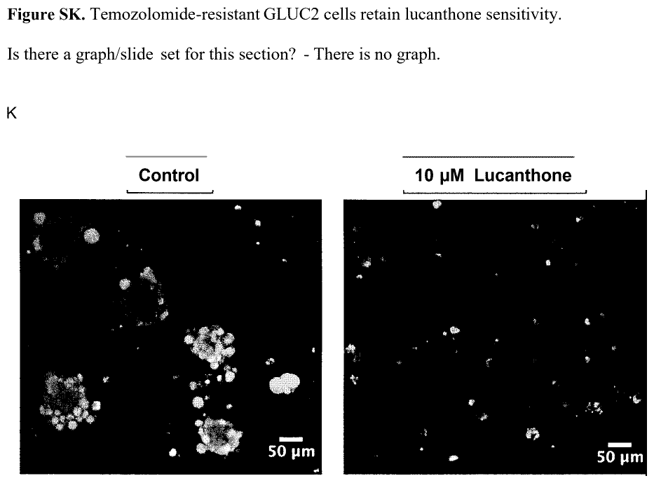

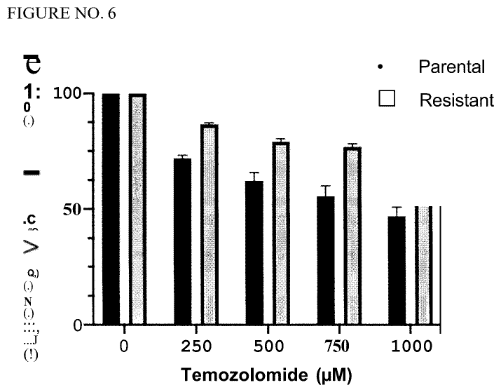

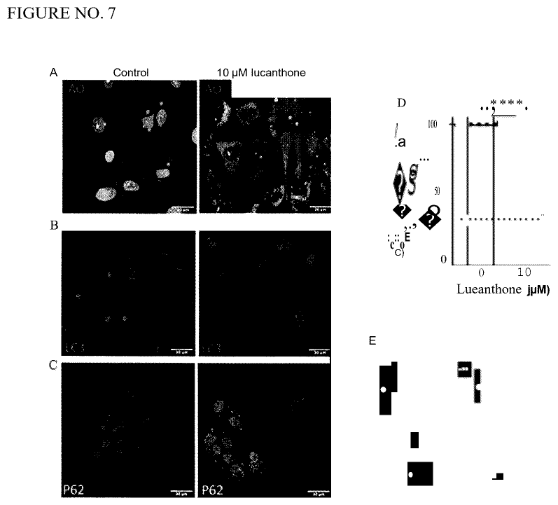

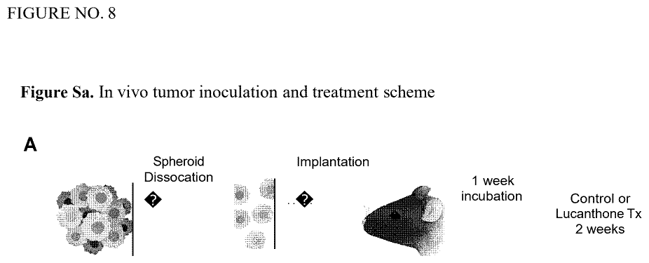

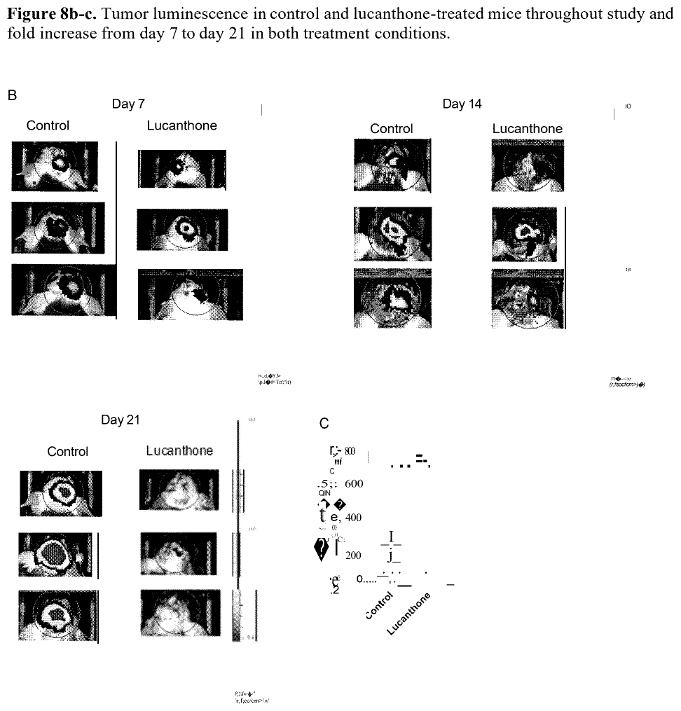

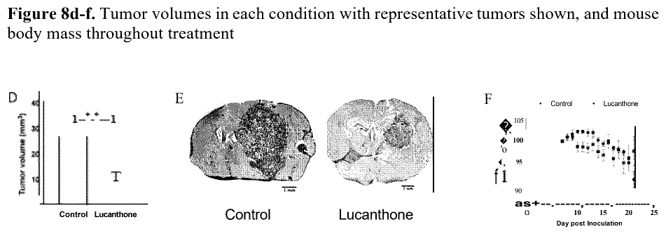

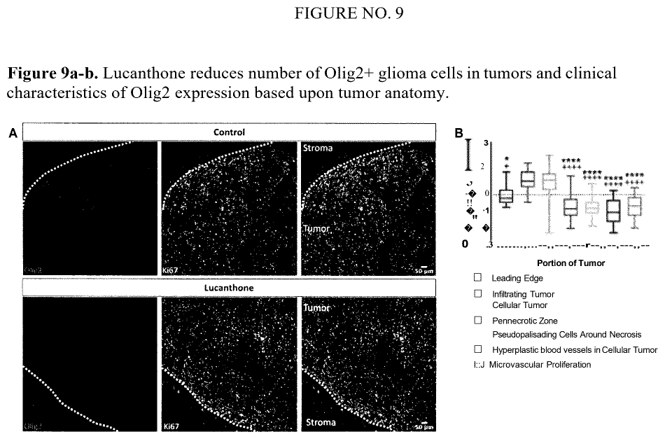

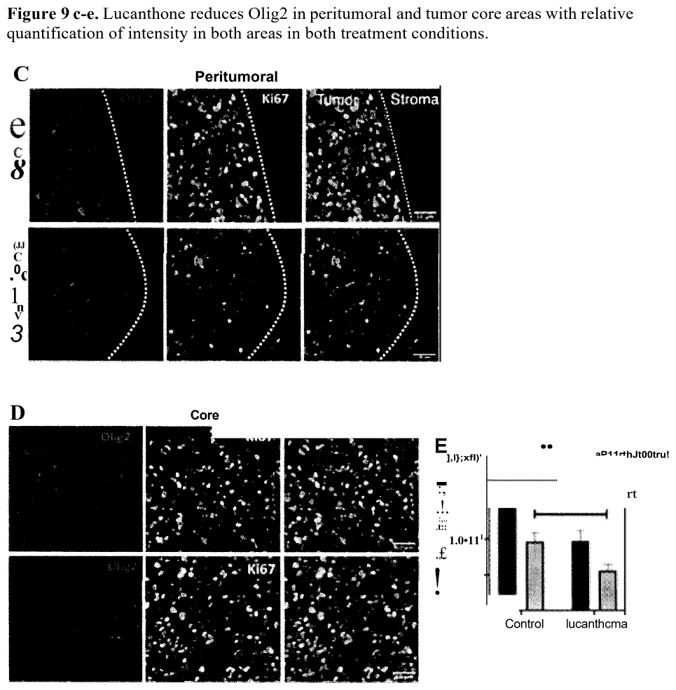

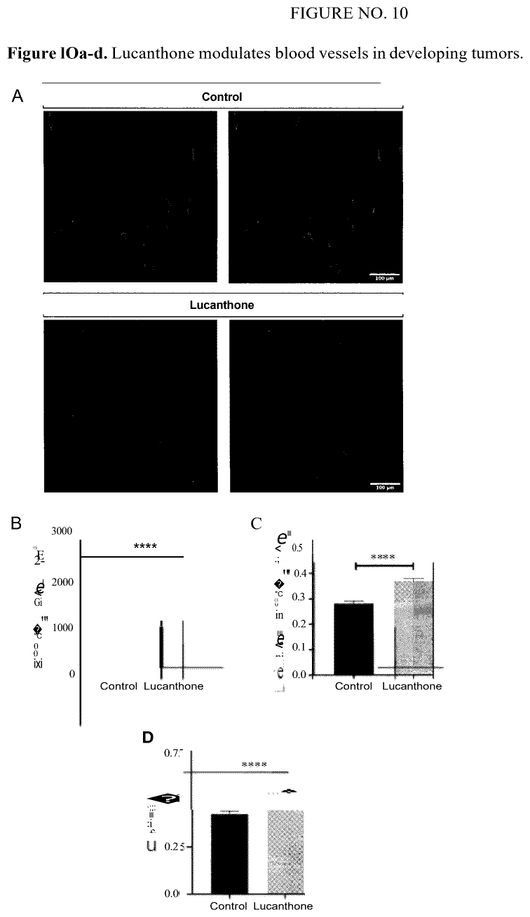

Figure_{[DR7J} 10 graphically demonstrates tumor microenvironmental changes induced by lucanthone. FIG. 10A Representative images of blood vessels marked by CD3 l of control- and lucanthone-treated tumors. Figure 1OB Blood vessel area. FIG. 10C Luminal area/blood vessel area. Figure l0D Blood vessel circularity. ****p<0.0001, Kolmogorov-Smirnov test. Bars are mean+/−SEM, N=4-5 animals per group. Figure l0E and l0F Representative images of Glutl levels in control- and lucanthone-treated tumors, respectively. Figure lOG Quantification of Glutl intensity in the tumor microenvironment. Bars are mean+/−SEM. N=5 mice **p<0.01, t-test. Figure l0H Glutl expression in necrotic areas in clinical specimens. Data adapted from the Ivy Glioblastoma Atlas. ****p<0.0001, Kruskal Wallis test. **p<0.01, ***p<0.001, ****p<0.0001, Dunn's test, compared to perinecrotic zone, ++++p<0.0001, Dunn's test, compared to pseudopalisading cells around necrotic areas. FIG. 101 CD8 cells in the tumor microenvironment in control- and lucanthone-treated tumors. *p<0.05, Mann-Whitney test Bars are mean+/−SEM, N=4 animals per group.

FIG. 11 illustrates how lucanthone increases yH2AX in tumors in vivo. Representative micrographs of tumor sections stained for the GAM marker F4/80 and yH2AX. Quantification of 7 animals per group. Bars are mean+/−SEM. **p<0.01, t-test.

FIG. 12 shows how lucanthone increases HSP60 levels in vivo. Representative micrographs of tumor sections stained for the mitochondrial marker HSP60. Quantification of 6-7 animals per group. Bars are mean+/−SEM. **p<0.01, Mann-Whitney test.

FIG. 13 illustrates how lucanthone compromises bEND.3 cell viability at high concentrations only. bEND.3 cells were treated with lucanthone for 72 hours and then an MTT assay was performed. Data are mean+/−SEM of 3 independent experiments. ANOVA, p<0.01, demonstrating a significant dose-response effect. *p<0.05, Sidak multiple comparison test.

FIG. 14 shows that levels of Glut4 are not affected by lucanthone treatment in vivo. Control- and lucanthone-treated tumors were stained for Glut4. Levels were similar among treatment groups.

FIG. 15 illustrates the effects of lucanthone on P2RY12 and TMEMl 19 levels in vivo. A) Control- and lucanthone-treated tumors were stained for P2RY12. Levels were similar among treatment groups; B) Representative micrographs of tumor core sections stained for TMEMl 19; C) Quantification of 5 animals per group. Bars are mean+/−SEM. *p<0.01, t-test.

FIG. 16 shows that TMEMl 19 marks glioma stem cells in vivo, not myeloid cells. Tumors inoculated in Macgreen-GFP mice were stained for multiple markers to identify which cells in the glioma tumor were indeed expressing TMEMl 19. A) TMEMl 19 does not co-localize with CSFl R-GFP-expressing myeloid cells in control tumors. B) A subset of Olig2-positive tumor cells stain positive for TMEMl 19. C) TMEMl 19-positive cells are not F4/80-positive, but F4/80-positive cells are also CSFlR-GFP-positive. D) A subset of TMEMl 19-positive cells are positive for the proliferation marker Ki67.

FIG. 17 demonstrates that lucanthone targets patient-derived glioma stem-like cells with greater potency over its hydroxylated metabolite, hycanthone. A) GBM43 cells cultured with serum and without serum. GBM9 cells grow in a similar way when cultured with or without serum. B. Western blot of CD44, P62, Sox2, LC3 and TMEMl 19 expression in adherent (adh) versus cancer stem cell (CSC) growth conditions. C) Comparison of lucanthone versus hycanthone effects on GBM43 and GBM9 stem-like cells. Data are mean+/−SEM of 3 independent experiments. ANOVA, p<0.01, demonstrating a significant dose-response effect. *p<0.05, **p<0.01, Dunn's multiple comparison test, compared to control. D) Calcein-AM stained micrographs of both cell lines treated with 0-3 uM lucanthone for 5 days.

FIG. 18 demonstrates that lucanthone inhibits spheroid formation at nanomolar concentrations in both patient-derived glioma cell lines. Lucanthone treatment at 400 nM and 800 nM dose-dependently reduces spheroid formation of glioma stem-like cells. ANOVA, p<0.0001, demonstrates a dose-dependent effect. ****p<0.0001, Dunn's multiple comparison test, compared to that of control treatment. Experiments were performed at least 6 times.

FIG. 19 shows that lucanthone inhibits autophagy in patient-derived gliorna stem-like cells. After 24 hours of 3 uM lucanthone treatment, GBM9 and GBM43 stern-like cells exhibit increased levels of P62 and LC3-II, demonstrating autophagy inhibition.

FIG. 20 illustrates how lucanthone targets lysosomes and damages mitochondria in patient-derived glioma stem-like cells. After 24 hours of 3 uM lucanthone treatment, GBM9 and GBM43 stem-like cells exhibit dilated lysosomes and significantly reduces DIOC63 staining. Mitotracker deep red (MT DR) marks mitochondria that are healthy or damaged, while DIOC63 marks healthy mitochondria.

FIG. 21 demonstrates that lucanthone complements effects of radiation and temozolomide in GBM9 and GBM43 cells. Data are mean+/−SEM of 4-6 independent experiments **p<0.01, t-test.

FIG. 22 shows that lucanthone is active against in GLUC2 stem-like cells that have recovered from 5 gy radiation treatment. Cells that recover from radiation treatment express CD133 but are still susceptible to anti-cancer effects of lucanthone, as shown by Calcein-AM stains and MTT assay. Data are mean+/−SEM of 4 independent experiments ANOVA, p<0.01, demonstrating a significant dose-response effect. *p<0.05, ****p<0.0001, Dunn's multiple comparison test, compared to control.

FIG. 23. Lucanthone is still active against EO771 triple-negative breast cancer cells that show brain metastatic tropism. Cells were isolated from the brain and were able to be cultured with and without serum. Cells cultured without serum formed spheres and were subject to lucanthone treatment. SEM of 6 independent experiments ANOVA, p<0.01, demonstrating a significant dose-response effect. **p<0.01, ****p<0.0001, Dunn's multiple comparison test, compared to control.

FIG. 24 graphically illustrates how lucanthone retains its activity even against glioma cells that have been selected for resistance to supraphysiological doses of temozolomide. A) Workflow of selecting GLUC2 cells for temozolomide sensitivity up to 500 uM doses. B) After cells were inoculated, in vivo imaging was used to stratify animals with similar disease burden on day 7. C) Relative increase of luminescence throughout treatment course in control and lucanthone-treated animals. D) Representative luminescent image intensities (6 animals per group). E) Representative tumor sections of control- and lucanthone-treated tumors. F) Quantification of tumor volumes. N=9 or more mice per group. Data for E are mean+/−SEM. *p<0.05. ***p<0.001. Student's t-test.

### DETAILED DESCRIPTION OF EXEMPLARY EMBODIMENTS OF THE INVENTION

The instant disclosure surprisingly teaches that the known anti-schistosomal agent lucanthone potently targets and inhibits CNS cancers, including glioblastomas. The novel findings presented herein demonstrate that lucanthone effectively kills glioma cells, at least in part by autophagy inhibition. Lucanthone additionally enhances temozolomide efficacy at sub-cytotoxic concentrations. Further unexpected results herein show that lucanthone potently suppresses growth of stem-like glioma cells, including temozolomide-resistant glioma stem cells. Correspondingly, lucanthone is highly effective at slowing tumor growth in vivo. Related studies demonstrate that lucanthone measurably reduces numbers of Olig2+ glioma cells in tumors, normalizes tumor vasculature, and prevents or diminishes tumor hypoxia. Thus, according to the teachings herein, lucanthone is demonstrated to be a potent new CNS cancer treatment agent that fulfills vital, unmet clinical needs, including by overcoming mechanisms of chemoresistance that have long rendered glioblastomas and other CNS cancers refractory to successful treatment.

Gliomas are the primary cancers of the central nervous system (CNS). Glioblastoma (GBM) is the highest grade, most aggressive and most common form of glioma in adults (1). Current standard of care therapy for GBM consists of maximum safe surgical resection followed by fractional radiation, chemotherapy (typically using the alkylating agent temozolomide (TMZ)) and adjunct treatment with tumor-treating fields (2). Median survival after diagnosis is around 16-20 months (2). Because GBM is ordinarily highly invasive, resection is usually incomplete, accounting for rapid recurrence and extraordinary lethality associated with this malignancy.

During GBM disease progression, patients often experience comorbidities, including drug resistant seizures, headaches, sleep disturbances and neurological deficits, in addition to the side effects of radiation and chemotherapy. The search for better GBM treatment agents and modalities has heretofore been complicated by the fact that most drug molecules cannot pass efficiently through the blood brain barrier, whereby many drug candidates that have shown in vitro efficacy have not proven useful in vivo, based on their inability to reach the brain.

Gliomas are comprised of multiple cell populations including glioma cancer stem cells (GSC), pericytes, infiltrating bone-marrow derived macrophages (BMDM) and microglia (3-5). In glioma, BMDMs and microglia accumulate in tumor tissue attracted by chemokines, such as CSFl and CCL2, secreted by tumor cells (6, 7) and constitute the glioma-associated macrophages/microglia (GAM). GAM promote or contribute to glioma cell survival, neoangiogenesis and immunosuppression in the tumor microenvironment (TME) (3, 4, 6, 7), which are all important target processes to inhibit in order for new drug compositions and methods to effectively manage GBM.

Previous reports have speculated that induction of autophagy in glioma cells may promote resistance to standard of care chemotherapies (8-11). Autophagic induction in tumor-associated pericytes and GAM has also been proposed to foster an immunosuppressive TME (5, 12). In addition, induction of autophagy has been reported to limit the oncolytic capacity of cytotoxic T-lymphocytes (CTL) in other tumors (13, 14). While these and other reports may help guide future research, clinically relevant benefits of autophagy-inhibiting drugs to augment CNS cancer therapies and control immunosuppression in the TME have yet to be demonstrated.

Lucanthone (marketed as Miracil D) is an anti-schistosome agent (15-20), known to inhibit topoisomerase II and AP endonuclease 1 (APEl) (21-24). Lucanthone has been reported to have some activity against solid tumors when paired with ionizing radiation (25), and to act in combination with TMZ against breast tumor cells in vitro (22, 26). Lucanthone has also been suggested to inhibit autophagy and lysosomal membrane permeabilization (27), possibly in a complementary manner with TMZ and radiation (22, 26). Lysosomal membrane permeabilization using chloroquine reportedly results in repolarization of tumor-associated macrophages from an immune-suppressive/pro-tumor ‘M2-like’ to an immune-promoting/anti-tumor ‘Ml-like’ phenotype (28). This phenotypic shift was also reported to correlate with an increase in pro-inflammatory markers (IFN-y, TNF-a, CD86, iNOS), a decrease in the expression of anti-inflammatory proteins (IL-10, Arg1) and increased anti-tumor T-cell immunity (28). Despite these and other studies, suggesting multiple contemplated effects of lucanthone for impacting tumor growth and activity, these proposed effects are complex and have heretofore remained poorly understood.

Prior reports suggesting anti-cancer properties of lucanthone fall far short of predicting any clinical or therapeutic use of this drug for controlling cancer. The extensive investigative results presented here resolve many uncertainties relating to lucanthone anti-cancer efficacy, and provide concrete guidance demonstrating clinical utility of lucanthone for treating CNS cancers in human subjects. The studies described herein employ multiple glioma cell lines to demonstrate that autophagy is actively and directly inhibited by lucanthone at sub-cytotoxic concentrations. In related studies, 1 uM lucanthone substantially complements and/or potentiates standard of care temozolomide anti-cancer effects in multiple mouse glioma cell lines. Further investigations herein demonstrate that enriched cancer stem cell sub-populations that express multiple sternness markers (SOX2, CD] 33, OLIG2 and/or Nestin) grown in 3D spheroids (an accepted model of the tumor microenvironment) are preferentially targeted and controlled by lucanthone (in comparison to “standard” glioma cell populations cultured in serum). The instant disclosure further demonstrates that lucanthone effectively treats intact 3D spheroids, reducing Olig2 activity/expression. Importantly, Olig2 appears to potentiate resistance/recurrence of CNS cancers following standard of care treatments. The surprising discovery here that lucanthone reduces Olig2 levels in accepted model systems of the glioma TME, indicates that lucanthone will be an important drug for both primary and adjunctive (follow-on to standard of care) treatments.

Applicant's investigations presented herein additionally treated glioma cells for several weeks with chemotherapy, revealing that after induction of the sternness marker CD133 (7), lucanthone potently targets and controls these refractory, sternness induced cells (comprising the most dangerous stem population of cells capable of mediating tumor recurrence). These surprising and unprecedented findings reveal that lucanthone represents a first in class drug for effective treatment of recurrent glioblastoma, a disease for which no prior drugs or treatment modalities have been shown to significantly prolong patient survival.

These ground-breaking discoveries by Applicant are further resolved and confirmed herein through extensive and conclusive in vivo studies, including investigations using live mammalian intracranial tumor models of glioblastoma. These subjects were inoculated with marked (luciferase expressing) tumor cells then effectively treated with lucanthone. This treatment began with lucanthone at 50 mg/kg, demonstrating a lack of apparent systemic toxicity at this high dosage. Tumor growth was monitored non-invasively by luminescent imaging for two weeks. In these novel studies, lucanthone potently mediated reduction in luminescent intensity, later shown to be correlated with significant decreases in tumor volume. Concomitant reductions in OLIG2+ glioma cancer stem cells were also observed in these subjects, indicating that lucanthone effectively targets and controls glioma tumor stem cells in vivo.

Yet additional work presented herein reveals that lucanthone mediates its surprising anti-CNS cancer effects in part by powerfully protecting and/or modifying the tumor microenvironment (TME), normalizing blood vessel architecture and function, reduce hypoxic stress and attendant cellular and tissue damage/dysfunction, and boosting and/or potentiating anti-cancer immunity (in particular, by protecting/promoting cellular anti-cancer immunity mediated by cytotoxic T lymphocytes (CTLs)). Previous reports suggested that chloroquine might reduce glioma hypoxia by normalizing tumor vasculature (27). However, like other previous studies discussed here, this study failed to provide substantiating in vivo data. The lengthy and detailed investigations herein demonstrate that blood vessels in lucanthone-treated tumors, in vivo, are smaller, yet they are more circular and provide higher luminal area relative to total vessel area—meaning they are structurally normalized and functionally enhanced for reducing hypoxic stress and damage (including impairment of immune function). Complementary studies herein show a surprising reduction in tumor hypoxia, marked by decreased levels of the glucose transporter Glutl (a protein induced in hypoxic environments by Hif2a), in lucanthone-treated tumors in vivo.

The extensive research data provided here demonstrate that lucanthone potently targets lysosomes and blocks autophagy in glioma cells at clinically relevant concentrations. Lucanthone is further demonstrated to mediate anti-lysosomal and anti-autophagy activities complementarily in combination with TMZ. Additionally, Applicant has discovered that lucanthone preferentially targets glioma stem cells in vitro, and can slow tumor growth in vivo, in clinically useful dosage forms and methods. Yet additionally, Applicant has shown that lucanthone normalizes tumor vasculature, reduces hypoxia and increase cytotoxic T cell infiltration into the core of glioma tumors in vivo—demonstrating potent and practical efficacy of this drug against glioma cells and tumors generally, including within the complex TME fostered by high-grade gliomas, and against chemotherapy-resistant and post-chemotherapy-recurrent gliomas.

Within clinical embodiments, the anti-CNS cancer lucanthone compositions and methods of the invention effectively treat CNS tumors, for example as demonstrated by reductions in tumor incidence, size, pathogenic progression, marker expression, and/or one or more cancer-associated disease diagnostic indicia or side effect(s). Lucanthone compositions and methods herein administered to CNS cancer patients will mediate substantial tumor volume reduction, or an observable reduction in tumor pathogenic status (e.g., as observed through biopsy or necropsy of existing tumors). Other diagnostic examples for determining clinical efficacy of lucanthone include a decrease in numbers of cells in treated versus control subjects expressing one or more cancer cell markers, or cancer stem cell markers (e.g., as determined by flow cytometry, Western blotting, or other methods using tumor biopsy, or patient blood sampling). In general, lucanthone treatment will effectively reduce one or more of these diagnostic indicators of reduced CNS cancer incidence or recurrence, slowed or reversed disease progression, reduced disease and/or treatment side effects, and/or improved disease status/health of treated subjects, by at least 5%, 10%. 25%, 30%, 50%. 75%. 90% or more compared to levels observed in placebo-treated control subjects. In illustrative embodiments, lucanthone will reduce CNS tumor size, neoplastic or metastatic disease status associated with tumor growth, new tumor formation, and metastasis, and/or levels of tumor-associated cancer stem cells by at least 25%, often by 50%-75% or greater, 90% or more, and up to 100% (e.g., to mediate long term remission, where patients remain free of detectable CNS cancer for 3-5 years or longer following end of treatment.

Novel aspects of the invention include clinical efficacy of lucanthone for selectively targeting and controlling CNS cancer stem cells, resolving a long unmet need for effective CNS cancer treatments that eradicate stem cells (in contrast to conventional chemotherapy methods that arguably “select for” stem cells and thereby leave the “treated” patient highly vulnerable to cancer recurrence). Well known assays, markers and labeling reagents can be routinely employed to demonstrate anti-stem cell efficacy of the lucanthone compositions and methods herein. Those skilled in the art will appreciate that such assays are readily designed and implemented to identify and quantify cancer stem cells, for example based on detection of positive stem cell markers (e.g., nestin, SOX2, Olig, CD15, CD133, SSEA-4, and others) using conventional assay technologies such as cytometry, immunobead capture, and immunocytochemistry. Employing these and related diagnostic targets and assays, lucanthone will reduce CNS cancer stem cells within patient tumors and/or in samples of patient blood and other tissues, by at least 20%, frequently by 30%-50% or more, up to levels of 80-100%, demonstrative of the potent clinical therapeutic use of lucanthone to prevent CNS cancers, including to prevent recurrence of CNS cancers, even aggressive high-grade glioblastomas, following failed, conventional chemotherapy treatment. More discrete assays will confirm that lucanthone mediates comparable percent reductions in tumor stern cell viability, proliferation capacity, tumor-initiation potential. and/or tumor promoting gene expression/differentiation lucanthone-treated and control subjects.

More generally, the clinical anti-CNS cancer effectiveness of lucanthone compositions and methods of the invention can be monitored and demonstrated by any combination of conventional oncological diagnostic methods, for example by tumor imaging with x-rays or MRI (e.g. to demonstrate that tumors have decreased in size and/or number in lucanthone-treated patients). Effectiveness will often be determined by radiographic or MRI observation of a decrease in tumor size. Effective lucanthone compositions and methods of the invention for treating CNS cancer will routinely yield at least a I 0%, 25%, 50%, 75%, 90% or greater reduction of tumor size in treated patients, or in average tumor size and/or number among a group of treated patients, compared to qualified, comparable control subjects.

Effectiveness of lucanthone anti-CNS cancer compositions methods of the invention against cancer, metastatic disease, and against stem cell viability/numbers/activity associated with cancer recurrence, will further be demonstrable by measuring circulating tumor cells, and or circulating cancer stem cells, in blood samples between suitable test and control subjects. This may be accomplished by any means applicable including, but not limited to immunomagnetic selection, flow cytometry, immunobead capture, fluorescence microscopy, cytomorphologic analysis, or cell separation technology. Effective anti-CNS cancer compositions and methods of the invention will routinely yield at least a 10%, 25%, 50%, 75%, 90% or greater reduction of circulating tumor cells generally, and/or or circulating cancer stem cells (expressing one or more diagnostic stem cell markers) in blood samples of treated patients, or among a group of treated patients, compared to qualified, comparable control subjects.

Effectiveness of lucanthone anti-CNS cancer compositions and methods of the invention relating to reduction of metastatic disease may further demonstrated by detecting/measuring primary tumor cell occurrence or number in secondary tissues or organs, including sites and structures in the CNS distant from a primary tumor site, and in rare cases non-CNS sites and tissues, such as bone, lymph nodes, liver and lungs, of treated versus control patients. Effective lucanthone anti-CNS cancer compositions and methods of the invention will yield at least a I 0%, 25%, 50%, 75%, 90% or greater reduction in the occurrence or number of primary tumor cells metastasized to other CNS sites and/or non-CNS secondary tissues or organs among treated patients compared to qualified, comparable control subjects.

In certain aspects of the invention, lucanthone compositions and methods for preventing and treating CNS cancer involve coordinate administration of an effective amount of lucanthone, along with a secondary treatment agent, treatment modality or treatment method. In certain exemplary embodiments, subjects are treated with lucanthone simultaneously or sequentially with a secondary treatment drug, agent or method, selected from: a chemotherapeutic drug (i.e., using a second anti-cancer or anti-metastatic drug, compound or chemical agent), radiation, chemotherapy. surgery, tumor-treating fields, or any combination of these secondary treatment agents/methods.

In certain “coordinate therapy” or ⋅‘combinatorial treatment” embodiments, the invention employs a lucanthone compound or pharmaceutical composition administered simultaneously (at the same time, optionally in a combined formulation) with a secondary drug, compound or chemical agent possessing combinatorial anti-cancer or anti-metastatic activity. Secondary chemotherapy drugs in this context are contemplated to broadly include agents classified as conventional chemotherapy drugs (for example taxanes); vascular disrupting agents (VDAs): IISP-90 inhibitors, immunotherapeutics, and many other classes of anti-cancer agents. Within these and related embodiments, lucanthone compound and secondary drug or treatment will be “combinatorially effective”, meaning biological activity (e.g., anti-cancer or anti-metastatic activity as defined herein), side effects, patient outcomes, or other positive therapeutic indicia will be improved over results observed in relevant control subjects treated with the lucanthone compound or composition alone, or the secondary drug alone.

Lucanthone compounds, compositions and methods of the invention can be coordinately employed with any of a range of secondary anti-cancer drugs, agents or interventions, in combinatorial formulations or coordinate treatment protocols (with lucanthone administered concurrently, prior or subsequent to the secondary treatment agent or method). In exemplary coordinate treatments, an anti-CNS cancer effective amount of lucanthone is administered coordinately with a chemotherapeutic drug or therapy. Chemotherapeutic drugs and therapies for secondary use within these aspects of the invention include anti-cancer and anti-hyperproliferative agents, agents that destroy or “reprogram” cancer cells, agents that modulate blood vessel growth associated with neoplasms, and many other classes of drugs harmful to neoplastic cellular targets. In this regard, useful chemotherapeutics and other anti-CNS cancer drugs within the invention include but are not limited to:
(1) Tubulin depolymerizing agents like taxoids such a paclitaxel and docetaxel;
(2) DNA damaging agents and agents that inhibit DNA synthesis;
(3) Anti-metabolites;
(4) Anti-angiogenics agents and vascular disrupting agents (VDAs);
(5) Antitumor antibodies and related immunologic drugs,
(6) Endocrine therapy;
(7) Immuno-modulators;
(8) Histone deacetylase inhibitors,
(9) Inhibitors of signal transduction;
(10) Inhibitors of heat shock proteins;
(11) Retinoids such as all-trans retinoic acid;
(12) Inhibitors of growth factor receptors or the growth factors themselves;
(13) Anti-mitotic compounds;
(14) Anti-inflammatory agents such as COX inhibitors, and
(15) Cell cycle regulators, check point regulators and telomerase inhibitors.

Within exemplary embodiments of the invention, lucanthone compositions and methods are administered coordinately with one or more, including any combination, of the following secondary anti-cancer (or adjunctive therapeutic) drugs, agents, methods and/or treatment modalities:
1. Temozolomide
2. Nitrosoureas like carmustine (BCNU) or lomustine
3. Radiation
4. Tumor treating fields (TTF)
5. Anti-PD-1/PD-Ll and/or anti-CTLA-4 therapy
6. CSF1R inhibitors
7. Pi3K/akt and/or mTOR inhibitors or drugs that converge on these enzymes
8. Drugs targeting receptor tyrosine kinases such as EGFR/FGFR/IGFlR
9. Levetiracetam (Keppra)
10. Riluzole/troriluzole
11. Biguanides like metformin or phenformin
12. Cannabinoids like THC or cannabidiol
13. Integrated stress response inhibitors such as ISRIB
14. TGFB receptor inhibitors
15. Val-083 (dihydrogalctitol)
16. Berubicin
17. 5-fluorouracil or other nucleotide/nucleoside analogues
18. Bevacizumab or other vascular disrupting agents
19. Drugs targeting microtubules
20. Connexin inhibitors
21. Drugs targeting tumor microtubes
22. Inhibitors of glutamate, dopamine, serotonin and other neurotransmitter receptors
23. Trans sodium crocetinate
24. Oscillating magnetic fields
25. Parp inhibitors
26. DNA repair protein inhibitors such as ATM/ATR inhibitors
27. Drugs that disrupt vascular/vasculogenic mimicry
28. Inhibitors of cyclin-dependent kinases such as CDK4/6 inhibitors and CDK2 inhibitors
29. Drugs targeting mitochondria such as mitophagy inducers

In related aspects of the invention, combinatorial methods and formulations are provided comprising lucanthone coordinately administered or admixed in a common dosage form with one or more secondary drugs, for example conventional chemotherapeutic drugs, along with one or more side-effect reducing therapeutics (e.g., anti-seizure drugs, antidepressants, antiemetics, pain drugs, etc., depend on what combinatorial therapy is being employed, e.g., chemotherapy versus radiation therapy).

Anti-CNS effective lucanthone compounds of the invention may be provided in a variety of forms and compositions, useful for administration by oral, topical, parenteral, transdermal, intravenous (iv) and other conventional routes. In useful pharmaceutical compositions, lucanthone will be provided in a pure or substantially pure form (e.g., at least 90%, 95%, or 98% purity), with minimal contaminants or byproducts (other than pharmaceutical carriers, fillers, delivery enhancing agents, excipients or other pharmaceutically acceptable additives). Effective lucanthone compounds of the invention include functionally comparable, equivalent or enhanced salts, prodrugs, metabolites, derivatives, analogs, complexes and conjugates of lucanthone, as well as rationally-designed chemical analogs of lucanthone having designed and tested side chain or other structural modifications (e.g., selected from deletions, substitutions and/or additions of chemical groups, side chains, linkers, coupled compounds, etc., that may enhance solubility, stability/half-life, lipophilicity, BBB penetration, cellular tropism, toxicity or other desired properties of the drug. Also useful are polymorphs, solvates, hydrates, metabolites and prodrugs of lucanthone compounds, analogs and derivatives.

Combinatorial efficacy of coordinate lucanthone therapies can occur for a variety of reasons, but is often attributed to complementary inhibition of one or multiple cancer cellular biological targets, processes or pathways. Individual targets, processes or pathways may provide “‘bypass” routes for targeted cells (e.g., cancer stem cells), requiring that multiple pathways be targeted to prevent the escape. When anti-CNS cancer stress is presented, for example using known anti-CNS cancer therapeutic drugs, certain cells, such as stem cells, may evade or bypass the disruption of normal tumor-associated targets/processes. For example, chemotherapeutics often induce hypoxic stress in a tumor microenvironment, which stress may be alleviated for certain cells in the TME by endogenous activity of heat shock (hs) proteins, for example heat shock factor 90 (Hsp90). Thus, in certain embodiments of the invention an I lsp90 inhibitor is coordinately administered with lucanthone to yield combinatorially effective clinical results. In other illustrative embodiments, lucanthone is coordinately administered with vascular disrupting agents (VDAs), with attendant benefits of allowing for lower VDA effective dosage and reduction of VDA-associated adverse side effects. Yet additional exemplary methods employ coordinate treatment with lucanthone and TMZ or other secondary anti-CNS cancer chemotherapeutic drug, such as taxanes. As described in detail within the examples below, lucanthone potently targets CNS cancer stem cells that “bypass” TMZ and other drugs through poorly understood resistance mechanisms.

The invention is further described for illustrative, non-limiting purposes by the Examples which follow. The skilled artisan will understand that the instant invention is not limited to the particular materials, process steps, or methods of design and use exemplified here, as these examples are provided for demonstrative not limiting purposes. Following the teachings of the invention as a whole, the invention can be adapted, optimized and expanded in equivalent form and purpose by the skilled artisan, without undue experimentation. Likewise, the terminology employed herein is exemplary to describe illustrative embodiments, not limit the scope of the invention.

### EXAMPLES

### Cell Culture

GL261 cells expressing luciferase (GLUC2) were obtained from the lab of Dr. Michael Lim. They are derived from a chemically induced astrocytoma in C57BL/6 mice (29). KR158 cells were obtained from the labs of Drs. Tyler Jacks and Behnam Badie, and are derived from genetically engineered Nfl/Tp53 mutants (30). Cells were maintained in DMEM, 10% serum, 1% antibiotic, 1% sodium pyruvate and incubated at 37° C. with 5% CO2. bEND.3 cells were cultured in DMEM with serum as above. Primary patient-derived human glioma cells (GBM43) which carries Nfl and Tp53 mutations, and GBM9, which carries Kras, Tp53, Rbl and PTEN mutations, were obtained from Dr. Jann Sarkaria at the Mayo Clinic from the xenograft cell line panel. To enrich for glioma stem-like cells (GSC) in GLUC2, KRISS GBM43 and GBM9 cells, serum was reduced step-wise over a week as described previously (31). EO771 murine triple-negative breast cancer cell that had metastasized to the brain were cultured in serum and serum-free conditions. GSC were cultured in serum-free DMEM medium containing F12 supplement along with pyruvate, antibiotics, N2 supplement, EGF, FGF and heparin (31).

### Crystal Violet Studies

For single lucanthone treatment studies, GLUC2 and KRISS cells were plated at a density of 2,000 and 1,000 cells per well, respectively, in a 6-well plate and allowed to adhere overnight. They were then treated with 10 μM lucanthone every 4 days for 12 days. On day 13, media were aspirated, and cells were fixed with 4% PFA for 10 minutes. Cells were then treated with 0.5% crystal violet solution for 20 minutes. Plates were washed and photographed.

For dual treatment studies (lucanthone and TMZ), GLUC2 and KR158 cells were plated at a density of 2,500 and 1,000 cells per well in a 12-well plate and allowed to adhere overnight. Cells were then treated with medium, TMZ, lucanthone, or a combination for 4 days. The media were aspirated, and the cells were washed with PBS once and incubated with standard medium for 3 days. The cells were fixed with PFA and treated with 0.5% crystal violet solution as above and photographed. Then lysis solution of 10% SDS in dH2O was added to the plates overnight. To quantify relative crystal violet intensity, the absorbance of the crystal violet-containing supernatant was read under a spectrophotometer at 590 nm with a reference wavelength of 670 nm. Data are graphed as percent of control (medium only-treated cells).,

### ,“t!TT Assay

Cells were plated in a 96-well plate and incubated overnight. Adherent tumor cells (2D cultures) were treated with lucanthone for 3 days and then subject to the MTT protocol as per manufacturer's instructions (Promega). GSC (3D cultures) were treated with lucanthone for 5 days, as this allowed sufficient time for spheroids to grow in culture. Prior to addition of the MTT reagent, plates were imaged under confocal microscopy with the addition of Calcein-AM and Ethidium homodimer to mark live cells and dead cells, respectively.

### Acridine Orange Stain

GLUC2, KRl 58 and GBM43 cells were plated on glass-bottom 35 mm plates overnight. They were then treated with medium or lucanthone for 48 hours. The cells were treated with 5 μg/ml acridine orange for 15 minutes. Plates were washed with PBS 3× and then incubated in complete medium. Plates were then imaged for acidic vesicle accumulation (5251590 nm) under confocal microscopy, according to manufacturer's instructions (Cayman chemical).

### Immunocytochemistry

For immunocytochemical analysis, GLUC2, KRl 58 and GBM43 cells were plated on glass coverslips overnight. Cells were treated with medium or lucanthone for 48 hours. The medium was aspirated and cells were fixed with 4% PFA for 10 minutes. Plates were then washed 3× with 0.3% TX-100 in PBS and wells were blocked with 3% normal goat serum/0.3% TX-100 in PBS for 1 hour. Cells were stained with primary antibodies overnight (LC3, Ki67, Nestin, Olig2, SOX2, CD133, p62, Cathepsin D, yH2AX). The primary antibody was removed, and cells were again washed 3× with 0.3% TX-100 in PBS after which time cells were incubated with fluorescent secondary antibodies for an hour at room temperature. Cells were then washed 3× with PBS, counterstained with DAPI and imaged under confocal microscopy. GSC were induced to adhere to glass slides by precoating glass slides with Geltrex for an hour.

### Western Blot

Immunoblotting was done as described previously (4). Briefly, cells were lysed in 50 mM Tris-HCl (pH 7.4) with 1% Nonidet P-40, 0.25% sodium deoxycholate, 150 mM NaCl, 1% SDS and 1 mM sodium orthovanadate. Proteins were denatured by boiling with treatment with BME. Proteins were run on SDS-page gels, transferred to PVDF membranes (Immobilon; Millipore). Membranes were washed with Tris-buffered saline with 0.1% Tween 20 and blocked in a 5% non-fat dry milk powder for 1 hour. Membranes were then probed for LC3 (1:1000), p62 (1:1000), Olig2 (1:1000), SOX2 (1:1000) CD44 (1:200), TMEMl 19 (1:1000) and B-Actin (1:2000; sigma Aldrich). Membranes were rinsed in TBS-T, probed with associated HRP-conjugated secondary antibodies and exposed to Pierce ECL substrate for 1 minute (Thermo Fisher Scientific) after which x-ray films were developed from membranes.

### RNA Isolation and Quantitative RT-PCR

To prepare RNA, GLUC2 spheroids were spun down and lysed with Trizol and processed usmg the manufacturers protocol. To obtain cDNA, one microgram of RNA was reverse transcribed on a Veriti thermocycler using the High Capacity cDNA Reverse Transcription Kit. Amplification was performed on a StepOnePlus real-time PCR machine using a SYBR green kit (Applied biosystems). Primer sequences are as follows: GAPDH forward, 5′-GCACAGTCAAGGCCGAGAAT-3′; GAPDH reverse, 5′-GCCTTCTCCATGGTGGTGGA-3′; Olig2 forward, 5′-CAAATCTAATTCACATTCGGAAGGTTG-3′; Olig2 reverse, 5′-GACGATGGGCGACTAGACACC-3′. GAPDH was used as an internal control.

### Radiation Experiments

To examine the interaction between lucanthone and ionizing radiation, 3000 GBM9 or GBM43 CSC were plated in a 96-well plate and allowed to adhere overnight. 1 hour prior to radiation, cells were treated with 1 or 3 uM lucanthone. Radiation was accomplished using a 100 kVp animal irradiator (Phillips RT-100) which irradiates at 0.75 gy per minute. After radiation, cells were incubated for 5 days, after which an MTT assay was performed.

### Limited Dilution Assay

To examine the effect of lucanthone on sphere-forming ability of patient-derived glioma stem-like cells, spheres were dissociated and then plated in 96 well plates at 200 or 400 cells per well, treated with lucanthone or control medium, and then allowed to grow in culture for two weeks. After this time, they were assessed for number of spheres formed.

### Animals

C57B16 mice were bred under maximum isolation on a 12:12 hour light: dark cycle with food ad libitum. MacGreen mice, expressing GFP under the CSflr promoter were genotyped prior to use according to our previous protocol (32)

### Murine Glioma Model

Gliomas were established in 3-4 month old male and female mice as described previously (3, 4, 33). GLUC2 GSC were dissociated with accutase and counted. Mice were anesthetized with 20 mg/kg avertin, a midline incision was made in the scalp, the skin retracted and a small burr hole was drilled in the skull at the following stereotactic coordinates from bregma: −lmm anteroposterior and +2 mediolateral. l×10⁵ GLUC2 GSC resuspended in PBS were injected over a period of 2 minutes at a depth of 3 mm. At the end of the injection, the needle was kept in the injection site for a further 3 minutes. After needle removal, the incision was sutured and mice were placed on a heating pad until they fully recovered from anesthesia. During the disease course if mice were found to have lost more than 15% of their initial body weight, they were euthanized. All animal procedures were approved by the Stony Brook University Institutional Animal Care and Use Committee.

### In Vivo Luciferase Imaging

GSC engraftment was visualized using the IVIS spectrum in vivo imaging system 7 days after inoculation and again on days 14 and 21. Briefly, mice were anesthetized using continuous isofluorane exposure. Their scalps were shaved. Mice were injected i.p. with 150 mg/kg D-Luciferin, carefully placed in the IVIS spectrum machine and imaged every 3-4 minutes for 40 minutes. Relative signal was quantified by technician blinded to experiment, and luminescence ratios of day 21 to day 7 were calculated to approximate disease progression throughout the course of treatment.

### Lucantlwne Treatment In Vivo

Lucanthone was solubilized in 10% DMSO, 40% HPCD in PBS. After confirming the presence of gliomas on day 7, mice were randomly divided to control and treatment groups, and treated with either saline or 50 mg/kg lucanthone i.p. every day from day 7 to day 20. On day 21, tumors were visualized by bioluminescent imaging, as above.

### lmmunohistochemistry

Mice were anesthetized with 20 mg/kg avertin and transcardially perfused with 30 ml PBS followed by 30 ml 4% PFA in PBS. Brains were removed and post-fixed in 4% PFA in PBS overnight. They were dehydrated for 48 hours in 30% w/v sucrose in PBS. Brains were then embedded in optimal cutting temperature compound (OCT, Tissue-Tek) and 20 μm coronal sections throughout the entire tumor were taken on a Leica cryostat (Nusslock, Germany) and collected on Superfrost plus microscope slides. To determine tumor volume, serial sections were taken from each animal and subjected to hematoxylin and eosin stain. Tumor volume was calculated as tumor area×20 μm thickness, x number of slides (34).

For immunohistochemical analysis, slides were brought to room temperature, washed 3× with 0.3% TX-100 in PBS and then blocked with 1% BSA/0.3% TX-100 in PBS for 1 hour. Slides were incubated overnight with appropriate primary antibodies (Table 1). The primary antibody was removed and slides were washed 3× 0.3% TX-100 in PBS and incubated with appropriate secondary antibodies for 1 hour. Slides were washed 3× with PBS, and counterstained with DAPI. Immunoreactivity was visualized by confocal imaging using the Leica SP8-x system, with white light and argon lasers.

TABLE 1
Antibodies used for immunocytochemistry and immunohistochemistry

| Antibody | Cat# | Dilution |
|---|---|---|
| Rabbit anti Cd133 | Novus, #NB2016518 | 1:500 |
| Rat anti CD31 | BD Biosciences, #557355 | 1:500 |
| Rabbit anti Glutl | Novus, #NB1 10-39113 | 1:200 |
| Rat anti CD8a | Invitrogen, #14-0081-82 | 1:50 |
| rat anti-Ki67, | Thermofisher, #14-5698-82 | 1:500 |
| Goat anti Cathepsin D | R&D systems, #AF1029 | 1:200 |
| Rabbit anti P62 | Novus, #NBP1-48320 | 1:200 |
| Rabbit anti LC3A/B | CST, #4108 | 1:200 |
| Mouse anti Nestin | Abeam, #AB1 1306 | 1:200 |
| Rabbit anti Olig2 | Millipore Sigma, #AB9610 | 1:500 |
| Mouse anti SOX2 | R&D systems, #MAB2018 | 1:500 |
| Goat-anti-rabbit Alexa Fluor 555 | Thermo, #A-21429 | 1:1000 |
| Goat-anti-rat Alexa Fluor 555 | Thermo, # A-21434 | 1:1000 |
| Goat-anti-rabbit Alexa Fluor 488 | Thermo, #A-11008 | 1:1000 |
| Donkey-anti-goat Alexa Fluor 555 | Lifetech, #A21432 | 1:1000 |

### Statistical Analysis

Data comparing two population means with a normal distribution were analyzed using Student's t-test. Data with non-normal distributions were analyzed using a Mann-Whitney test. Differences in cumulative distributions were assessed with the Kolomogorov-Smirnov test. To assess for synergistic interactions, King's synergy test was used (35-37). Blood vessel circularity was calculated using the equation Circularity=4*lf*(areal(perimeter²)). Alpha value was set at 0.05 prior to starting experiments. Power analysis was used to determine the appropriate number of animals used in each experiment. Experiments were replicated with the two tumor lines. Statistical analysis was performed using Graphpad Prism (Graphpad Software Inc, La Jolla, CA).

### Lucanthone Effectively Targets Lysosomes and Inhibits: Autophagy¹_{[DR8J}

To examine whether lucanthone affects the growth of the two murine glioma cell lines GLUC2 and KR158, lucanthone (FIG. 1A) was added to glioma cultures at 10 μM every 4 days for 2 weeks (FIG. 1B), which reflects concentrations observed in the serum of patients (26). The proliferation of both cell lines was hindered. To investigate whether the possible mechanism by which lucanthone acts on glioma cells engaged autophagy, glioma cells were treated with lucanthone for 48 hours, and then stained them with acridine orange, which accumulates in acidic vacuolar organelles and shifts from green to red fluorescence (37). In control conditions, only few lysosomes were present in the cell lines. After treatment with lucanthone, cultures in both cell lines exhibited a remarkable diffuse cytoplasmic staining of dilated lysosomes (FIG. 1C) with a corresponding increase in LC3 punctae (Figure lD). These data parallel observations after treatment with chloroquine in other tumor types (38) and support the conclusions here that lucanthone potently targets lysosomes and affects autophagic function at clinically relevant concentrations.

To elaborate Levels of the autophagy cargo receptor p62 and Cathepsin D were also assessed. P62 accumulates in cells in which autophagy has been functionally inhibited and Cathepsin D is a lysosomal aspartyl protease (27). These data demonstrate that after 48 hours of lucanthone treatment, P62 and Cathepsin D increase in both glioma cell lines, with a higher relative increase of both proteins in KR158 cells (Figure lE, j1FJDR9J), further demonstrating lucanthone's potent activity to inhibit autophagy at clinically relevant concentrations.

To examine whether lucanthone acts as an inhibitor of topoisomerase 2 or APEl, lucanthone induction of DNA damage was determined in glioma cell lines. GLUC2 and KR158 cells were treated with lucanthone for 48 hours, after which levels of yH2AX, a DNA damage marker, were assessed (39). As a positive control, glioma cells were also treated with the FDA-approved topoisomerase 2 inhibitor etoposide. While etoposide produced a marked increase in yH2AX intensity, lucanthone only produced a minimal effect, indicating it is exerting the above-observed effects primarily through autophagy inhibition. When levels of cleaved caspase-3 were evaluated, only minimal induction of cleaved caspase-3 in GLUC2 and KR158 spheroids treated with 10 μM lucanthone for 48 hours were observed, indicating that lucanthone may not be inducing apoptosis in these glioma cell lines (FIG. 2, as was determined for another autophagy inhibitor, thymoquinone (which induces cathepsin-mediated, but caspase-independent cell death) (40).

### Lucanthone Unexpectedly Enhances Temozolomide Efficacy

The interaction between lucanthone and TMZ was investigated by performing combination studies in vitro. First, MTT assays were performed to determine minimally effective concentrations of lucanthone in both cell lines. Lucanthone exerted a dose-dependent reduction in cell viability, with an IC50 of approximately 11-13 μM (FIG. 3A). Two-way ANOVA illustrated that both cell lines were similarly sensitive to lucanthone, implying that this drug may be useful regardless of driver mutations. These data also pointed towards the use of 1 μM lucanthone for the combination studies, since this concentration exerted minimal effects alone on both cell lines.

It has been reported that GL261 and KRl 58 cells exhibit striking resistance to TMZ in vitro (41, 42). To evaluate this phenomenon, GL261 and KR158 cells were treated with control medium, lucanthone or TMZ alone, or both drugs for 4 days, and then cultures were allowed to recover for 3 days before analysis. In this extended treatment format, 1 μM lucanthone alone, or 50 μM TMZ or 100 μM TMZ produced only a modest effect on GL261 and KR158 cells (FIG. 3B, C). However, crystal violet intensity was markedly decreased when cells were treated with a combination of lucanthone and TMZ (FIG. 3B, 3C, p<0.05, King's synergy test). Our data, in agreement with previous studies on breast tumor cells (22), suggest that even lower doses of lucanthone may be useful when paired with standard of care therapies to slow glioma progression.

To clarify how lucanthone augments anti-tumor effects of TMZ, changes in the levels of yH2AX, a marker of DNA damage, were evaluated in both cell lines. After 48 hours, changes in yH2AX intensity were evident in cultures treated with TMZ, but not in those treated with lucanthone (FIG. 3D-3E (DRIOJ). Cultures treated with both drugs exhibited slightly increased levels of yH2AX compared to cultures treated with TMZ alone, but this abbreviated study did not show the increase to be statistically significant.

### Lucanthone Targets Glioma Stem Cells to Prevent Acquired Temozolomide Resistance

Cancer stem cells are progenitor-like tumor cells that repopulate the tumor after what is considered “successful” treatment, driving tumor recurrence and fatality. It is now accepted that cancer stem cells (termed here GSC) are rapidly dividing (43) and resistant to both TMZ and radiation (44, 45). Recent data reveal that GSC preferentially rely on autophagy for their survival and resistance to TMZ (46, 47). To investigate this phenomenon, sternness characteristics were induced in GLUC2 and KR158 cell lines as described above. Both glioma cell lines grew as partially suspended spheroids. 1 week after culturing cells in sternness medium, GLUC2 spheroids stained positive for the sternness markers nestin, SOX2 and Olig2, while KRl 58 spheroids stained positive for nestin, CD133 and SOX2 (FIG. 4). Cells staining positive for these markers also stained positive for the proliferation marker Ki67, demonstrating these cells are indeed actively proliferating. Additionally, western blot analyses indicate the vGLUC2 spheroids express higher levels of SOX2 and Olig2, while KR158 spheroids express higher levels of SOX2 than their adherent counterparts (FIG. 4).

After determining that these cells expressed sternness markers, they were treated with increasing concentrations of lucanthone. Remarkably, doses as low as 3 μM produced a strong oncolytic effect in these GSC. Lucanthone reduced spheroid area in both cell lines (Figure SA, B). Further, treatment with lucanthone in a dose-dependent manner resulted in reduced numbers of spheroids formed in culture and reduced viability of the cultures (Figure SC, D). These data show that lucanthone may preferentially kill cells left behind after treatment with modalities such as TMZ and radiation. Additionally, the IC50 of lucanthone was approximately 2 μM for KR158 and GLUC2 GSC. This is in contrast to an IC50 of 11-13 μMin cells cultured with serum. These data indicate that stem-like glioma cells may be more susceptible than other glioma cells to autophagy inhibition by lucanthone.

To gain mechanistic insight into how lucanthone reduces sternness, KR158 and GLUC2 GSC were cultivated to form spheroids for 10 days, then spheroids were treated with 10 μM lucanthone for 48 hours and assayed for alterations in levels of LC3 and p62. By western blot analysis, we observed that lucanthone increased p62 levels in GLUC2 and KR158 GSC and increased levels of LC3-II as well (Figure SE, 30). These data reveal that lucanthone acts in a similar manner in these studies as observed in adherent 2D cultures. Notably, in control conditions LC3 punctae were also observed in spheroids, suggesting a higher level of baseline autophagy in GSC and a higher reliance on autophagy in general. In addition to assessing for changes in autophagic flux, we assessed for changes in the levels of sternness markers after treatment. A strong reduction in Olig2 intensity was evident in lucanthone-treated cultures (Figure SH-31), while expression on nestin and SOX2 did not change. Using RT-qPCR lucanthone reduced Olig2 mRNA expression in GLUC2 spheroids by >60% (FIG. 51), with some detectable change also observed in Ki67 in these cultures.

Despite multimodal treatment, the recurrence rate for glioblastoma is ˜100%. It has been proposed that glioma cells change throughout the course of treatment, such that cells surviving treatment may be functionally different than the parental tumor (48-50). To elucidate this question, the ability of lucanthone to mediate oncolytic effects on glioma cells selected for their ability to resist the standard chemotherapy temozolomide, TMZ was analyzed. GLUC2 cells were treated with two cycles (48 hours of treatment and 7 days recovery per cycle) of 250 μM TMZ and 3 cycles of 500 μM TMZ. After this selection, surviving cells started forming spheres in serum-containing medium, similar to the ones observed when these cells were cultured in sternness-promoting medium. These spheroids expressed the prototypic sternness gene CD133 whereas parental GLUC2 spheroids did not (Figure SJ), suggesting that glioma cells dynamically respond to genotoxic therapy by acquiring stem-like morphology and characteristics (49). Cells selected for TMZ resistance were also less sensitive to TMZ treatment than parental GLUC2 cells (FIG. 6). In spite of becoming more stem-like, these cultures were still markedly sensitive to 10 μM lucanthone (Figure SK), indicating lucanthone can effectively slow growth of TMZ-resistant malignant glioma cells.

To demonstrate that lucanthone can effectively target human glioma cells, patient-derived glioma cells were obtained from the Mayo Clinic (GBM43 cells), bearing Tp53 and Nfl mutations. After treatment with lucanthone, GBM43 cells exhibited a similar acridine orange cytoplasmic staining pattern as seen in GLUC2 and KR158 cells (FIG. 7A). Additionally, LC3 and P62 were detectably increased (FIGS. 7B and 7C), indicating autophagy was effectively inhibited by lucanthone in these cells. After enriching for stem-like qualities in these cells, treatment with 10 μM lucanthone completely inhibited spheroid formation in these cultures and drastically reduced cell viability (FIG. 7D-7E). Together these data show that lucanthone potently inhibits autophagy in both mouse and human glioma cells.

### Lucanthone Therapeutically Inhibits Glioma Growth In Vivo

To assess translational potential, the efficacy of lucanthone was investigated in a mouse model of glioma. GLUC2 GSC were allowed to form spheroids for 10 days in culture. The spheroids were mechanically dissociated and 100,000 GLUC2 cells were implanted in the striatum of mice. Tumors were allowed to form for 7 days. Tumor cell presence was confirmed using IVIS imaging system on day 7, after which mice were segregated into two groups: one group received saline every day until day 21 while the other group received 50 mg/kg lucanthone every day until day 21. The animals were imaged on days 14 and 21 (FIG. 8A). On day 14, 5 of the 7 control mice exhibited a 2-fold increase in luminescence. In contrast, only 1 of 8 lucanthone-treated mice experienced a two-fold increase in luminescence, suggesting that lucanthone mitigated tumor growth between days 7 and 14 (chi-squared test, p<0.05). By day 21, control (saline)-treated glioma-bearing mice experienced a ˜200-fold increase in tumor luminescence compared to day 7, whereas lucanthone-treated mice experienced only a 10-fold increase in tumor luminescence (FIG. 8B, SC). Upon histological analysis, the tumors of lucanthone-treated mice were approximately 60% smaller than those of saline-treated animals (Figures SD, SE). Moreover, saline-treated mice experienced cachexia (Figure SF), whereas lucanthone-treated mice did not experience significant weight loss throughout the course of treatment (Figure SF).

### Lucanthone Reduces Olig2+ Glioma Cells In Vivo

Standard of care therapies for glioma enrich for tumor stem-like cells, which may play a key role in glioma recurrence (44, 45). The instant investigations focus on how lucanthone may target and modulate glioma stem cells in vivo. The expression of sternness genes such as Olig2 and SOX2 was assessed in experimental tumors. Initial examination revealed that the density of Olig2+ cells was highest near the periphery of tumors (FIGS. 9A-9D), with a significant number of Olig2+ cells observed near the tumor core as well. These data are consistent with reports that Olig2+ glioma cells are present at increased numbers near the tumor periphery (51). According to the Ivy Glioblastoma Atlas, an anatomically annotated transcriptional dataset of human glioblastoma tumors (52), Olig2 expression is increased in areas of infiltrating tumor and cellular tumor, and reduced in areas of necrosis and around blood vessels (FIG. 9B). These findings indicate spatial expression of Olig2, GLUC2 GSC observed here models that observed in human disease.

In contrast to the abundant Olig2 expression observed in saline-treated mice, a striking reduction in Olig2 positivity was noted around the periphery of lucanthone-treated tumors and near the core of these tumors. Two-way ANOVA revealed that in both treatment conditions, Olig2 intensity is higher near the tumor border, and that lucanthone resulted in reduction of Olig2 intensity at the tumor periphery and in the tumor core (FIG. 9E). Ki67 positivity was similar in both treatment conditions. Additionally, SOX2 expression was not significantly different between treatment conditions, which parallels the result when individual spheroids were treated with lucanthone in vitro. While modulation of yH2AX by lucanthone in these in vitro did not rise to a level of statistical significance, yH2AX positivity was increased in vivo in lucanthone-treated tumors (FIG. 11). Increases in yH2AX were apparently restricted to glioma cells, as most of the cells that exhibited increases in yH2AX were not staining for the GAM marker, F4/80 (FIG. 11). While lucanthone may not induce direct, significant increases in yH2AX intensity in vitro as seen with etoposide treatment, long-term lucanthone treatment is predicted to act on topoisomerase II and/or APEl in vivo to increase DNA damage. Additionally, lucanthone is predicted to induce DNA damage in vivo indirectly. As an autophagy inhibitor, lucanthone is expected to inhibit turnover of damaged mitochondria (mitophagy), which will result in persistence of mitochondria releasing reactive oxygen species in glioma cells, inducing DNA damage. Increased levels of HSP60 are noted here in lucanthone-treated tumors (FIG. 12), a heat shock protein which marks mitochondria (53).

### Lucanthone Mediates Therapeutic Changes in the Glioma Tumor Microenvironment (TME), Including Normalization of Vasculature in the TME

In addition to demonstrating tumor-cell specific effects of lucanthone in vivo, the effects of lucanthone on other cell types in the tumor microenvironment (TME) were examined. In addition to directly targeting tumor cells, chloroquine (a reported autophagy inhibitor) normalized formation of blood vessels in the TME by directly acting on endothelial cells (54). Chloroquine augmented Notchl signaling in endothelial cells, and as a consequence, reduced blood vessel tortuosity and increased blood vessel patency. The studies here below show that lucanthone and chloroquine exert blood vessel normalizing effects in the TME by similar mechanisms, and that lucanthone is an effective drug candidate for normalizing blood vessel formation in developing gliomas.

Glioma tumor sections were stained for CD31, an endothelial cell marker. Blood vessel area, luminal area and overall blood vessel circularity were assessed. Large blood vessels were observed in control tumors (though many exhibited a small luminal area), with many vessels appearing tortuous with reduced circularity. In lucanthone-treated tumors, the blood vessels were smaller but showed substantially increased luminal areas, and were less tortuous and more circular. These observations indicate that lucanthone potently affects angiogenesis in the TME, acting directly on endothelial cells (FIGS. 10A-1OD). CD31 intensity was also diminished in lucanthone-treated tumors (Figures lOA-lOD). To examine if lucanthone acted directly on endothelial cells, bEND.3 cells were treated with lucanthone for 72 hours. Lucanthone exerted a dose-dependent effect on the cells, significantly reducing bEND.3 cell viability at 20 μM concentration, following incubation for 72 hours (FIG. 13).

To further elucidate the vascular normalizing activity of lucanthone, tumor hypoxia was assessed. In addition to proteins such as Hifl a/Hif2a, there are multiple other proteins induced in areas of tumor hypoxia, including Carbonic Anhydrase IX (CAIX) and Glutl (55). Tumors in both treatment conditions displayed little CAIX positivity, while control-treated tumors displayed remarkable Glut1 positivity, particularly in necrotic tumor areas (Figure 1OE). Lucanthone-treated tumors specifically displayed minimal Glutl positivity (Figure 1OF). Quantification of Glutl intensities is shown in FIG. 10G. Glutl expression in control tumors also mirrors expression patterns observed clinically (Figure 1OH). While Glutl was reduced throughout the tumor, another glucose transporter, Glut4, was also expressed throughout the tumor in saline- and lucanthone-treated conditions (FIG. 14), suggesting glucose transporter expression is not globally affected. These data indicate that, in addition to exerting potent tumor-cell specific effects, lucanthone modulates additional targets and processes in the TME.

### Lucanthone Therapeutically Modifies the Glioma TME by Protecting and/or Increasing Numbers of Anti-Tumor Effective Cytotoxic T Cells

The data above, showing that lucanthone normalizes blood vessels in the TME of CNS tumors is important for a number of clinically relevant reasons, including the correlative finding that lucanthone decreases hypoxic stress and damage to cells in the TME. An important cell type contemplated as beneficiaries of these cyto-protective actions of lucanthone, include immune, endocrine and other cells responsible for mediating a host of anti-tumor activities in healthy subjects. By reducing hypoxia in gliomas through improved blood vessel structure-function, lucanthone is predicted to increase the efficacy of radiation therapy, and to protect populations and activity of immune and immune-signaling cells, including most importantly, cancer-killing cytotoxic T cells.

To demonstrate lucanthone's anti-CNS cancer, immune cell-protective and immune-enhancing effects, CD8a+ cytotoxic T lymphocytes (CTLs) in TME of animal glioma model subjects were analyzed and quantified in control- and lucanthone-treated tumors. The findings from these studies (see, e.g., FIG. 101) demonstrate that, correlated with lucanthone's activity of normalizing tumor vasculature and reducing hypoxia, lucanthone increases cytotoxic T cell infiltration into, and/or survival in, the tumor core. A substantial increase in numbers of cytotoxic T cells was observed in the center of tumors of treated versus control mice, indicating that lucanthone effectively relieves immunosuppression in the TME of gliomas at clinically useful concentrations (Figure 1OI).

The demonstration here, that lucanthone potently counteracts glioma tumor pathogenesis, by normalizing blood vessels, reducing hypoxic stress in the TME, and promoting activity/survival of anti-CNS cancer CTLs, establishes that lucanthone will be a clinically effective agent to treat CNS tumors, particularly useful for high-grade glioma treatment, and against chemotherapy resistant and post-chemotherapy recurrent CNS tumors presenting a complex and treatment-challenging TME.

### Lucanthone Therapeutically Modulates Lysosomes and Autophagy in the Glioma TME, and Specifically Targets and Controls Glioma Stem Cells

Targeting lysosomes in a clinically effective manner involves activity against multiple cell types in the glioma microenvironment, including glioma-associated macrophages/microglia (GAM). To elucidate these activities of lucanthone, differences in myeloid cell populations were evaluated by staining for P2RY12 and TMEMl 19. P2RY12+ cells appeared mainly around the rim of gliomas in both treatment conditions (FIG. 15). However, TMEMl 19+ cells were detected throughout control-treated tumors, and to a lesser extent in lucanthone-treated tumors (FIG. 15B, C). TMEMI 19 staining patterns were distinct from those of P2RY12, suggesting TMEMI 19 marks a distinct population of cells.

To clarify this concept, GLUC2 GSC tumors of mice established on the Macgreen background (in which myeloid cells, including macrophages and microglia endogenously express GFP) were stained for TMEMl 19. Surprisingly, TMEMl 19+ cells did not colocalize with GFP+ myeloid cells. TMEM119+ cells were enriched toward the border of these gliomas, similar to the pattern observed with Olig2. To further elucidate this spatially specific staining pattern, the tumors were co-stained for Olig2 and TMEMl 19, showing that a significant amount of Olig2+ cells also stained positive for TMEMl 19 (FIG. 16). These cells did not co-localize with GFP+ myeloid cells. Additionally, a significant portion of the TMEMl 19+ cells were Ki67. Finally, an established marker of myeloid cells, F4/80 was tested, which faithfully marked GFP+ myeloid cells. However, cells that were TMEM119+ did not co-localize with F4/80, providing further evidence that TMEMl 19 marks a sub-population of glioma cells. This is believed to be the first discovery that TMEMl 19 is expressed on glioma cells in vivo. TMEMI 19 is expressed on breast tumor stem cells, where it reportedly enhances sternness by activating the Wnt/ [symbol] -catenin pathway (56). Collectively these findings and reports evince a new and surprising discovery, that lucanthone effectively target and control TMEMl 19+ glioma stem cells.

### Lucanthone Targets Patient-Derived Glioma Stem-Like Cells and Acts Complementary to Temozolomide and Ionizing Radiation to Effectively Control CNS Cancers

Yet additional studies herein show that lucanthone targets patient-derived glioma stem-like cells. GBM43 and GBM9 stem-like cells. Patient-derived cells grown in culture with serum form adherent monolayers, but when grown in serum-deprived conditions with EGF and FGF form 3-dimensional spheroids (FIG. 17A). GBM9 and GBM43 spheroids exhibit higher levels of the sternness genes CD44 and SOX2, illustrating greater malignant potential (FIG. 17B). Also, the spheroids exhibit higher levels of TMEM119 (FIG. 17B), showing that TMEMll 9 is indeed expressed by mouse glioma cells and by patient-derived glioma cells, highlighting a novel subset of glioma cells that can be targeted by lucanthone. In spheroids, p62 and LC3-II is decreased showing, increased autophagy in these stem-like cells and a potential dependence on autophagy. As well, lucanthone preferentially inhibits the growth of these cells versus that of its hydroxylated metabolite hycanthone. Lucanthone exerts an IC50 of about 1.5 uM in both cell lines, whereas hycanthone exhibits decreased potency. 3 uM hycanthone reduces viability to a lesser extent than that of lucanthone (FIG. 17C, FIG. 17D).

Additional studies show that lucanthone reduces sphere-forming capacity of GBM43 and GBM9 cells in culture at sub-micromolar concentrations. 400 nM lucanthone reduces cells' ability to form spheres by 70-85% while 800 nM lucanthone reduces cells' capacity to form spheres by >95% (FIG. 18). This suggests even low doses of lucanthone may be used to prevent recurrence. After 24 hours of 3 uM lucanthone treatment, both cell lines exhibited an increase in lapidated LC3 (LC3-II) and p62, suggesting lucanthone inhibits autophagy in these cells to a similar extent to that of murine glioma cell lines (FIG. 19). Cellular studies illustrate that lucanthone induces lysosome dilation, as evidenced by larger lysosomes imaged via lysotracker red (LT red) and also damages mitochondria, as shown by the substantial reduction in the DIOC63 staining pattern (FIG. 20). Additionally, we also show that lucanthone augments the efficacy of radiation in both patient-derived cell lines. In GBM9 cells, lucanthone and radiation alone exert minimal effects, but together reduce viability by −45%. In GBM43 cells, radiation actually increases viability when administered alone. Lucanthone treatment alone reduces viability by 15%, but combination treatment reduces viability by about 30% (FIG. 21). In both cell lines, 75 uM temozolomide minimally affect viability of cultures, but this effect is strongly potentiated by addition of 1 uM temozolomide. These data show that lucanthone can augment effects of DNA damaging modalities, either by chemotherapy or radiation (FIG. 21).

From a different perspective, we treated GLUC2 cells with 5 gy radiation and cultured cells for 7 days to allow for a fraction of cells to undergo programmed cell death. Cells that survived expressed the sternness/recurrence marker CD133 (FIG. 22). Cells were then exposed to lucanthone, which notably still retained its potency and efficacy in these radiation-resistant tumor cells (FIG. 22), still exhibiting an IC50 well below 3 uM (FIG. 22).

In addition to studies with human and murine glioma cells, experiments were performed on murine EO771 triple-negative breast tumor cells that had metastasized to the brain. EO771 cells were isolated from the brains of mice, dissociated and put into culture. Cells grew as a monolayer with the addition of serum (FIG. 23), and as spheres without serum. Though these cells exhibited metastatic and aggressive behaviors in vivo, they were still susceptible to lucanthone, with an IC50 of 2 uM (FIG. 23). These data show that lucanthone may be able to target cells of peripheral organs/tissues that metastasize to the CNS, in addition to cancers that arise in the CNS.

### Lucanthone Slows the Growth of Glioma Stem-Like Cells That Have Been Selected for Resistance to High-Dose Temozolomide

Further studies interrogate whether lucanthone has the ability to slow the growth of gliomas that exhibit acquired resistance to temozolomide. Currently, there exist no therapies to extend patient survival after glioma recurrence. To that end, GLUC2 cells were treated with escalating doses of temozolomide for several months until cells rebounded from multiple doses of 500 uM temozolomide, a dose far exceeding what is clinically achievable (FIG. 24A). Cells were then implanted into the striatum of mice. After 7 days, mice with similar disease burden were stratified into control or lucanthone treatment groups (FIG. 24B). After approximately 3 weeks of lucanthone treatment, relative luminescent increases (correlative of disease burden) decreased by about 50% in the lucanthone treatment group (FIG. 24C). Representative images are shown before animal sacrifice (FIG. 24D). Tumor volume was also found to be reduced by 50% as a result of lucanthone treatment (FIG. 24E, FIG. 24F). This data demonstrates that glioma cells that overcome temozolomide therapy may be amenable to lucanthone treatment and would pave a way to extend the survival of patients who inevitably will experience glioma recurrence.

The foregoing detailed studies and definitive findings clearly establish that lucanthone selectively targets and controls glioma stem cells in vivo, in a manner and at dosages evincing clinical efficacy to reduce morbidity and recurrence of CNS cancer, including glioma, in human subjects. The studies presented here utilize an art-accepted model predictive of human glioma drug efficacy, including murine stage 3 and stage 4 glioma models, with varied tumor sizes, TME changes and invasion into neighboring, healthy brain regions. Additional studies will further clarify the potent utility of lucanthone for targeting and controlling glioma stem cells, by specifically tracking changes in glioma cellular expression of genes associated with disease progression and recurrence (SOX2, OLIG2, CD133, NANOG, OCT4, c-myc) (1-4). To further clarify the cellular targets and processes affected by lucanthone treatment, expanded studies of numerous glioma/glioblastoma cell lines are in progress. These studies will further demonstrate dose-dependency pertaining to all diverse lucanthone activities disclosed herein. Within these ongoing investigations, cellular targets and processes associated with CNS cancer progression are being evaluated. In one arm of these studies, lucanthone treatment across a diverse panel of cell lines, over a period of 24-96 hours, will be analyzed for effects on phosphorylated (activated) and total levels of cancer-associated proteins STAT3, Pi3k, akt, MAPK, ERK.1/2, RAS, Raf, MEK, mTOR and others. All of these protein targets are implicated to have roles in mediating radiotherapy and chemotherapy resistance, aberrant cell proliferation, invasion and metastasis in CNS cancers. Additional aspects of the ongoing studies are focused on detecting lucanthone-mediated modulations in endoplasmic reticulum stress, alterations in redox homeostasis (reactive oxygen species) and effects on mitochondrial autophagy.

In more detailed aspects of Applicant's continuing investigative work, lucanthone's effects on growth pathway activity and P-glycoprotein levels and iNOS are being studied. P-glycoprotein is a drug efflux pump that removes chemotherapeutic drugs from cancer cells. Its expression is regulated by PI3K/akt/mtor activity. Ongoing studies will determine lucanthone's ability to modulate p-glycoprotein levels and/or function, which may reveal yet additional potential for lucanthone to combinatorially treat CNS cancers in combination with conventional therapies and drugs (e.g., carmustine). Carmustine (BCNU), is another drug used to treat GBM. It is removed quickly from cells by p-glycoprotein, and much of the drug remaining after this process is chemically modified by nitric oxide synthase. Based on the findings herein and preliminary findings from ongoing studies, lucanthone may act to impair drug efflux and/or chemical modification of drugs by targeted cancer cells, to increase the efficacy of carmustine and other CNS cancer drugs by multiple mechanisms. Additional, related studies are focused on detecting lucanthone-mediated changes in levels and/or activities of DNA repair proteins, including AMT, ATR and RAD51. Within these continuing studies, lucanthone is being investigated in combination with a large panel of additional, secondary cancer drugs, including carmustine, VAL-083, and berubicin, among others, using standard glioma cells cultured in 2D culture with serum, and CNS cancer stem cells cultured in 3D culture with growth factors.

Parallel, expanded in vivo studies are also in preparation to implant GLUC2 cancer stem cells in mice in experimental groups: Group 1 receiving saline, Group 2 radiation, Group 3, lucanthone, Group 4 lucanthone and radiation, Group 5 lucanthone and a VEGF antagonist, Group 6 lucanthone and a PDl antagonist (immunotherapy), and Group 6 lucanthone combined with one or more secondary CNS cancer drug(s) (e.g., TMZ, carmustine, VAL-083, and/or berubicin). These studies comprehend parallel histopathology, immunohistochemistry and survival analyses, from which the solo and combinatorial anti-cancer effects of lucanthone will be further determined. Flow cytometry will be conducted on test and control tumors to determine lucanthone-mediated effects on immune cell composition of the treated and untreated tumors. Related histopathology studies will involve sectioning tumors of treated and control mice for histology and immunohistochemistry to detect spatially localized, lucanthone-mediated alterations in number of cancer stem cells, tumor vasculature, hypoxia (by assessing Glutl levels), immune cell numbers/activity, and expression/activity of an array of cancer growth, activity, metastatic potential, immune cell/function, and sternness markers. A corresponding panel of studies will expand all of these arms to incorporate human glioma cells introduced into athymic mice.

Yet additional studies are in preparation to develop novel formulations of lucanthone in a liposomal composition, including liposomal nanoparticulate formulations, that will enhance delivery, tropism and bioavailability of lucanthone dosage forms for CNS tumors.

### Clinical Efficacy of Lucanthone for Treatment of Glioblastoma

Patients newly diagnosed with glioma will be selected for a first set of clinical lucanthone studies. Admitted patients will be qualified for tumor expression of unmethylated MGMT promoter (meaning the subject's tumors produce MGMT protein and are more likely to respond to temozolomide). Subjects will be grouped as follows: Group 1, control group, with treatment limited to surgical resection; Group 2 subjects will be treated with surgical resection plus lucanthone therapy (250 mg tablets orally t.i.d. for a total daily lucanthone dosage of 750 mg); Group 3 will be treated with surgery plus standard carmustine therapy (implanted in a time-release wafer at the surgical site; Group 4 will receive surgery plus standard TMZ therapy; Group 5 will receive surgery plus standard ionizing radiation therapy; Group 6 will be treated with surgery plus carmustine plus lucanthone therapy; Group 7 will receive surgery plus TMZ plus lucanthone therapy; Group 8 will receive surgery plus ionizing radiation plus lucanthone therapy. A parallel set of studies will be conducted for patients with recurrent glioblastoma following failed conventional treatment (surgery and chemotherapy).

Test and study subjects will be monitored responses and outcomes, determined by assessing standard diagnostic indices of tumor biology, disease progression, stabilization and reduction, at 6, 12, 18, and 24 months, with continuing follow-up monitoring at 36, 48 and 60 month. Primary diagnostic indices will include survival generally, along with recurrence- and progression-free survival. Secondary diagnostic indicia will comprehend the full range of pathological, histological, and immunohistochemical indicators (i.e., all patient disease criteria, pathological indices, cellular processes and marker) as described herein and otherwise known in the oncological arts, to assess CNS cancer growth, tumor morphology and activity, disease progression or regression, and all other indicia of disease status and treatment efficacy described herein above, among test and control subjects. For example, blood samples will be taken and peripheral blood mononuclear cells (PBMCs) isolated before treatment initiation and at milestone points during the course of the lucanthone treatment studies. These and other samples will be analyzed according to the description herein, for example to detect increases in LC3 autophagosomes, in the cargo receptor P62, in stem cells and sternness markers, along with comprehensive panel of other markers of CNS cancer disease presence, activity, progression and remission, as described above and otherwise known in the art.

The foregoing description and examples show that lucanthone compositions and methods of the invention arc powerful therapeutic tools for treating CNS cancers, including high-grade glioma, despite many obstacles documented previously. Lucanthone overcomes the general obstacle of identifying effective drugs that are deliverable and active across the blood-brain barrier (BBB), which has evolved to exclude large and charged molecules from accumulating in the CNS. Extensive research has been conducted over many years to identify drugs for targeting glioma that do not present unacceptable adverse side effects associated with current standard of care genotoxic stressors. In general, novel and repurposed drug candidates tested to date, having low BBB transit capacity, have limited clinical use, even for candidates that exhibited potent therapeutic effects in model systems (57). Further complicating these efforts, the role of GSC in gliomas, with their marked resistance to standard therapies including radiation and TMZ treatment, contributes to the high rates recurrence and poor prognosis of this disease, even after aggressive treatment with current standard of care interventions (44, 45).

The surprising data presented here show that lucanthone, a drug utilized for the treating schistosomal infections, inhibits autophagy in glioma cells when administered systemically, and slows the growth of intracranial gliomas in vivo. These data show unequivocally that lucanthone enters the brain at therapeutic levels, and can act either against CNS cancers either as a monotherapy or in concert with existing therapies (e.g., with temozolomide chemotherapy, radiation, and/or surgery).

Most interventions tailored to treating high-grade gliomas minimally prolong patient survival. Gliomas are almost universally resistant to treatment using TMZ, radiation, angiogenesis inhibitors, and tumor-treating fields, which resistance mechanisms has been contemplated to be mediated at least in part by cytoprotective autophagy (8, 9, 11, 44, 47, 58-61). Chloroquine and hydroxychloroquine have been proposed as potential autophagy inhibitors for certain cancers, but for glioma chloroquine exhibits poor penetration of the blood-brain barrier (62) and low potency (27). The instant studies demonstrate that lucanthone is a potent autophagy inhibitor, that is well tolerated in the clinical setting. Additionally, lucanthone can transit the blood-brain barrier, where it is effective at sub-cytotoxic concentrations, alone or in combination with TMZ, to treat glioma, and specifically to target and control glioma stem cells, thereby reducing or preventing disease recurrence.

Lucanthone has been shown to act as a topoisomerase II poison as well as an APE1 inhibitor at high concentrations. The surprising findings shown here, however, show that the primary function of lucanthone against CNS cancers is to disrupt autophagy. After treatment, the data here show extensive accumulation of autophagosomes in both KR158 and GL261 cells, further demonstrating that lucanthone exerts its effects independent of driver mutations. It is of particular interest that when glioma cells were cultured in sternness-promoting conditions, they exhibited increased sensitivity to lucanthone at doses as low as 3 μM. Since GSC are notoriously resistant to standard treatments, the development of adjuvant therapies that target a resistant sub-population may be useful in managing this disease and preventing recurrence. Lucanthone may preferentially target this sub-population by inducing lysosomal membrane permeabilization (LMP). The data presented here demonstrate that after lucanthone treatment, Cathepsin D is found throughout the cell, which is likely due to lysosomal rupture and release of lysosomal contents into the cytoplasm. Prior reports indicate GSC are susceptible to LMP (63-65), confirming that interfering with lysosomal function can effectively target cells spared from standard glioma treatments. The data here also show that lucanthone targets glioma cells CD133+ glioma cells having acquired resistance to TMZ (previous reports link TMZ-treatment to conditioning/selection of glioma cells to acquire more stem-like characteristics (49)). Because there are no therapies currently approved for treating recurrent glioblastoma, the invention herein provides extraordinary clinical benefits by treating glioblastoma subjects having acquired resistance to temozolomide (and/or ionizing radiation).

Lucanthone's inhibitory effect on sternness was further illustrated herein by investigating changes in LC3, and the sternness markers nestin, SOX2 and Olig2. As an initial observation, there were noticeable numbers of autophagosomes in control-treated spheroid cultures, indicating that GSC are more reliant on autophagy for survival at baseline conditions. However, there was a significant reduction in the number of cells in spheroids that stained positive for Olig2. In triple-negative breast tumor cells with constitutively active STAT3, the autophagy inhibitor chloroquine reduces active STAT3 (66). In glioma, inhibiting STAT3 activation by pharmacological or genetic means has been shown to reduce Olig2 levels (67), observations that may tie together lucanthone's mechanism with the observed reduction in Olig2. These in vitro results were recapitulated in vivo: Tumors derived from control-treated mice exhibited robust Olig2 intensity, especially at the tumor border. Lucanthone reduced Olig2 levels at the border and core of the tumors (FIG. 6). Olig2+ glioma cells exhibit increased resistance to standard therapies (68, 69), further encouraging the concomitant use of lucanthone with aforementioned interventions.

Gliomas exhibit dysregulated angiogenesis, which may contribute to the development of tumor hypoxia. Chloroquine was previously shown to act on endothelial cells in the melanoma tumor microenvironment. Chloroquine decreased the degradation of endothelial Notch 1, which functions to normalize tumor blood vessels and increases perfusion of the tumor. The studies here show that blood vessels of tumors treated with lucanthone are profoundly “normalized” (i.e., they exhibit increased circularity and reduced tortuosity). Decreasing tumor hypoxia may serve multiple functions, including increasing the delivery of systemic therapies to the whole tumor mass. In addition, eliminating pockets of hypoxia in gliomas through proper vessel perfusion could increase the efficacy of radiation therapy (70, 71) and restore the activity of cytotoxic T cells (72).

The advent of immunotherapies in the clinical setting has sparked an interest in understanding the role of both the innate and adaptive immune systems in the progression of aggressive tumor types, such as high-grade gliomas. Gliomas are comprised of multiple cell types specific to the CNS, and are heavily composed of CNS-resident microglia and blood-derived macrophages (73). Offsetting the tumor-promoting functions of these cells may directly slow the growth of gliomas and interact favorably with TMZ (48, 74, 75) and radiation (76. Investigations in peripheral tumor types, such as melanoma and hepatocellular carcinoma, revealed that late-stage autophagy inhibition with chloroquine, which was shown to act as an inhibitor of palmitoyl-protein thioesterase 1 (Pptl) (77-79), reverses the immunosuppressive nature of tumor-associated macrophages and thus increases the efficacy of T-cell targeted PD-1 therapies (28, 77). While direct protein target(s) of lucanthone to mediate autophagy inhibition are yet uncertain, the structural similarity between lucanthone and chloroquine suggests that Pptl, TopII and Apel are potential lucanthone targets. Considering that lucanthone appears to augment T cell infiltration into the glioma microenvironment, lucanthone emerges here as a strong candidate for modulating pro- and anti-tumorigenic functions of glioma-associated microglia and macrophages, alone and in combination with targeted therapies such as PD-I inhibitors and radiation.

Taken together, the data here evince that lucanthone will effectively treat CNS cancers, including recurrent/TMZ-resistant high-grade gliomas in human subjects. Additionally, lucanthone acts in a complementary or synergistic (coordinate) manner with existing glioblastoma therapies, including TMZ and radiotherapy, which complementary effects are unexpectedly mediated through direct effects on glioma cells, and by additional effects on endothelial cells and glioma stem cells. Combining lucanthone with DNA-damaging therapies, radiation and other immune-stimulating therapies will thus yield potent new therapeutic tools for clinical use.

Although the foregoing invention has been described in detail by way of example for purposes of clarity of understanding, it will be apparent to the artisan that certain changes and modifications may be practiced within the scope of the appended claims which are presented by way of illustration not limitation. The invention will thus be understood not to be limited, except in accordance to the claims which follow or may later be presented for examination. Various publications and other references have been cited with the foregoing disclosure for economy of description. Each of these references is incorporated herein by reference in its entirety for all purposes.

### REFERENCES

1. A. Omuro and L. M. DeAngelis: Glioblastoma and other malignant gliomas: a clinical review. JAMA, 310(17), 1842-50 (2013) doi:10.1001/jama.2013.280319
2. R. Stupp, S. Taillibert, A. Kanner, W. Read, D. Steinberg, B. Lhermitte, S. Toms, A. Idbaih, M. S. Ahluwalia, K. Fink, F. Di Meco, F. Lieberman, J. J. Zhu, G. Stragliotto, D. Tran, S. Brem, A. Hottinger, E. D. Kirson, G. Lavy-Shahaf, U. Weinberg, C. Y. Kim, S. H. Paek, G. Nicholas, J. Bruna, H. Hirte, M. Weller, Y. Palti, M. E. Hegi and Z. Ram: Effect of Tumor-Treating Fields Plus Maintenance Temozolomide vs Maintenance Temozolomide Alone on Survival in Patients With Glioblastoma: A Randomized Clinical Trial. JAMA, 318(23), 2306-2316 (2017) doi:10.100l/jama.2017.18718
3. J. T. Miyauchi, M. D. Caponegro, D. Chen, M. K. Choi, M. Li and S. E. Tsirka: Deletion of Neuropilin 1 from Microglia or Bone Marrow-Derived Macrophages Slows Glioma Progression. Cancer Res, 78(3), 685-694 (2018) doi:10.1158/0008-5472.CAN-17-1435
4. J. T. Miyauchi, D. Chen, M. Choi, J. C. Nissen, K. R. Shroyer, S. Djordevic, I. C. Zachary, D. Selwood and S. E. Tsirka: Ablation of neuropilin 1 from glioma-associated microglia and macrophages slows tumor progression. Oncotarget, 7(9), 9801-9814 (2016) doi:10.18632/oncotarget.6877
5. R. Valdor, D. Garcia-Bernal, D. Riquelme, C. M. Martinez, J. M. Moraleda, A. M. Cuervo, F. Macian and S. Martinez: Glioblastoma ablates pericytes antitumor immune function through aberrant up-regulation of chaperone-mediated autophagy. Proc Natl Acad Sci USA, 116(41), 20655-20665 (2019) doi:10.1073/pnas.1903542116
6. A. L. Chang, J. Miska, D. A. Wainwright, M. Dey, C. V. Rivetta, D. Yu, D. Kanojia, K. C. Pituch, J. Qiao, P. Pytel, Y. Han, M. Wu, L. Zhang, C. M. Horbinski, A. U. Ahmed and M. S. Lesniak: CCL2 Produced by the Glioma Microenvironment Is Essential for the Recruitment of Regulatory T Cells and Myeloid-Derived Suppressor Cells. Cancer Res, 76(19), 5671-5682 (2016) doi:10.1158/0008-5472.CAN-16-0144
7. S. M. Pyonteck, L. Akkari, A. J. Schuhmacher, R. L. Bowman, L. Sevenich, D. F. Quail, 0. C. Olson, M. L. Quick, J. T. Huse, V. Teijeiro, M. Setty, C. S. Leslie, Y. Oei, A. Pedraza, J. Zhang, C. W. Brennan, J. C. Sutton, E. C. Holland, D. Daniel and J. A. Joyce: CSF-lR inhibition alters macrophage polarization and blocks glioma progression. Nat Med, 19(10), 1264-72 (2013) doi:10.1038/nm.3337
8. S. A. Abdul Rahim, A. Dirkse, A. Oudin, A. Schuster, J. Bohler, V. Barthelemy, A. Muller, L. Vallar, B. Janji, A. Golebiewska and S. P. Niclou: Regulation of hypoxia-induced autophagy in glioblastoma involves ATG9A. Br J Cancer, 117(6), 813-825 (2017) doi:10.1038/bjc.2017.263
9. Y. S. Hori, R. Hosoda, Y. Akiyama, R. Sebori, M. Wanibuchi, T. Mikami, T. Sugino, K. Suzuki, M. Maruyama, M. Tsukamoto, N. Mikuni, Y. Horio and A. Kuno: Chloroquine potentiates temozolomide cytotoxicity by inhibiting mitochondrial autophagy in glioma cells. J Neurooncol, 122(1), 11-20 (2015) doi:10.1007/sl 1060-014-1686-9
10. T. Kanzawa, I. M. Germano, T. Komata, H. Ito, Y. Kondo and S. Kondo: Role of autophagy in temozolomide-induced cytotoxicity for malignant glioma cells. Cell Death Differ, 11(4), 448-57 (2004) doi:10.1038/sj.cdd.4401359
11. H. Ye, M. Chen, F. Cao, H. Huang, R. Zhan and X. Zheng: Chloroquine, an autophagy inhibitor, potentiates the radiosensitivity of glioma initiating cells by inhibiting autophagy and activating apoptosis. BMC Neural, 16(1), 178 (2016) doi:10.1 186/s12883-016-0700-6
12. J. Xu, J. Zhang, Z. Zhang, Z. Gao, Y. Qi, W. Qiu, Z. Pan, Q. Guo, B. Li, S. Zhao, X. Guo, M. Qian, Z. Chen, S. Wang, X. Gao, S. Zhang, H. Wang, X. Guo, P. Zhang, R. Zhao, H. Xue and G. Li: Hypoxic glioma-derived exosomes promote M2-like macrophage polarization by enhancing autophagy induction. Cell Death Dis, 12(4), 373 (2021) doi:10.1038/s41419-021-03664-1
13. L. DeVorkin, N. Pavey, G. Carleton, A. Comber, C. Ho, J. Lim, E. McNamara, H. Huang, P. Kim, L. G. Zacharias, N. Mizushima, T. Saitoh, S. Akira, W. Beckham, A. Lorzadeh, M. Moksa, Q. Cao, A. Murthy, M. Hirst, R. J. DeBerardinis and J. J. Lum: Autophagy Regulation of Metabolism Is Required for CD8(+) T Cell Anti-tumor Immunity. Cell Rep, 27(2), 502-513 e5 (2019) doi:10.1016/j.celrep.2019.03.037
14. X. Liang, M. E. De Vera, W. J. Buchser, A. Romo de Vivar Chavez, P. Loughran, D. Beer Stolz, P. Basse, T. Wang, B. Van Houten, H. J. Zeh, 3rd and M. T. Lotze: Inhibiting systemic autophagy during interleukin 2 immunotherapy promotes long-term tumor regression. Cancer Res, 72(11), 2791-801 (2012) doi:10.1158/0008-5472.CAN-12-0320
15. W. Alves: Further studies on the treatment of urinary bilharziasis with lucanthone hydrochloride. Bull World Health Organ, 18(5-6), 1109-11 (1958)
16. S. Archer: Recent progress in the chemotherapy of schistosomiasis. Prag Drug Res, 18, 15-24 (1974) doi:10.1007/978-3-0348-7087-0 3
17. S. Archer, K. J. Miller, R. Rej, C. Periana and L. Fricker: Ring-hydroxylated analogues of lucanthone as antitumor agents. J Med Chem, 25(3), 220-7 (1982) doi:10.1021/jm00345a006
18. S. Archer, A. H. Zayed, R. Rej and T. A. Rugino: Analogues of hycanthone and lucanthone as antitumor agents. J Med Chem, 26(9), 1240-6 (1983) doi:10.1021/jm00363a007
19. D. A. Berberian, H. Freele, D. Rosi, E. W. Dennis and S. Archer: Schistosomicidal activity of lucanthone hydrochloride, hycanthone and their metabolites in mice and hamsters. J Parasitol, 53(2), 306-11 (1967)
20. D. M. Blair, F. Hawking and et al.: Miracil; clinical trial on patients infected with Schistosoma haematobium and S. mansoni. Br J Pharmacol Chemother, 4(1), 68-80 (1949) doi:10.1111/j.1476-5381.1949.tb00517.x
21. M. D. Naidu, R. Agarwal, L. A. Pena, L. Cunha, M. Mezei, M. Shen, D. M. Wilson, 3rd, Y. Liu, Z. Sanchez, P. Chaudhary, S. H. Wilson and M. J. Waring: Lucanthone and its derivative hycanthone inhibit apurinic endonuclease-1 (APEl) by direct protein binding. PLoS One, 6(9), e23679 (2011) doi:10.1371/journal.pone.0023679
22. M. Luo and M. R. Kelley: Inhibition of the human apurinic/apyrimidinic endonuclease (APE1) repair activity and sensitization of breast cancer cells to DNA alkylating agents with lucanthone. Anticancer Res, 24(4), 2127-34 (2004)
23. L. Dassonneville and C. Bailly: Stimulation of topoisomerase II-mediated DNA cleavage by an indazole analogue of lucanthone. Biochem Pharmacol, 58(8), 1307-12 (1999) doi:10.1016/s0006-2952(99)00221-x
24. R. E. Bases and F. Mendez: Topoisomerase inhibition by lucanthone, an adjuvant in radiation therapy. Int J Radiat Oneal Biol Phys, 37(5), 1133-7 (1997) doi:10.1016/s0360-3016(97)00113-2
25. S. Turner, R. Bases, A. Pearlman, M. Nobler and B. Kabakow: The adjuvant effect of lucanthone (miracil D) in clinical radiation therapy. Radiology, 114(3), 729-31 (1975) doi:10.1148/114.3.729
26. J. D. Del Rowe, J. Bello, R. Mitnick, B. Sood, C. Filippi, J. Moran, K. Freeman, F. Mendez and R. Bases: Accelerated regression of brain metastases in patients receiving whole brain radiation and the topoisomerase II inhibitor, lucanthone. Int J Radiat Oneal Biol Phys, 43(1), 89-93 (1999) doi:10.1016/s0360-3016(98)00374-5
27. J. S. Carew, C. M. Espitia, J. A. Esquivel, 2nd, D. Mahalingam, K. R. Kelly, G. Reddy, F. J. Giles and S. T. Nawrocki: Lucanthone is a novel inhibitor of autophagy that induces cathepsin D-mediated apoptosis. J Biol Chem, 286(8), 6602-13 (2011) doi:10.1074/jbc.Ml 10.151324
28. D. Chen, J. Xie, R. Fiskesund, W. Dong, X. Liang, J. Lv, X. Jin, J. Liu, S. Mo, T. Zhang, F. Cheng, Y. Zhou, H. Zhang, K. Tang, J. Ma, Y. Liu and B. Huang: Chloroquine modulates antitumor immune response by resetting tumor-associated macrophages toward Ml phenotype. Nat Commun, 9(1), 873 (2018) doi:10.1038/s41467-018-03225-9
29. J. I. Ausman, W. R. Shapiro and D. P. Rall: Studies on the chemotherapy of experimental brain tumors: development of an experimental model. Cancer Res, 30(9), 2394-400 (1970)
30. K. M. Reilly, D. A. Loisel, R. T. Bronson, M. E. McLaughlin and T. Jacks: Nfl;Trp53 mutant mice develop glioblastoma with evidence of strain-specific effects. Nat Genet, 26(1), 109-13 (2000) doi:10.1038/79075
31. L. Yi, C. Zhou, B. Wang, T. Chen, M. Xu, L. Xu and H. Feng: Implantation of GL261 neurospheres into C57/BL6 mice: a more reliable syngeneic graft model for research on glioma-initiating cells. Int J Oneal, 43(2), 477-84 (2013) doi:10.3892/ijo.2013.1962
32. J. T. Miyauchi, D. Chen, M. Choi, J. C. Nissen, K. R. Shroyer, S. Djordevic, I. C. Zachary, D. Selwood and S. E. Tsirka: Ablation of Neuropilin 1 from glioma-associated microglia and macrophages slows tumor progression. Oncotarget, 7(9), 9801-14 (2016) doi:10.18632/oncotarget.6877
33. M. D. Caponegro, K. Oh, M. M. Madeira, D. Radin, N. Sterge, M. Tayyab, R. A. Moffitt and S. E. Tsirka: A distinct microglial subset at the tumor-stroma interface of glioma. Glia, 69(7), 1767-1781 (2021) doi:10.1002/glia.23991
34. H. Zhai, S. Acharya, I. Gravanis, S. Mehmood, R. J. Seidman, K. R. Shroyer, K. A. Hajjar and S. E. Tsirka: Annexin A2 promotes glioma cell invasion and tumor progression. J Neurosci, 31(40), 14346-60 (2011) doi:I 0.1523/JNEUROSCI.3299-11.2011
35. V. Kumar, D. Radin and D. Leonardi: Probing the Oncolytic and Chemosensitizing Effects of Dihydrotanshinone in an In Vitro Glioblastoma Model. Anticancer Res, 37(11), 6025-6030 (2017) doi: I 0.21873/anticanres.12049
36. V. Kumar, D. Radin and D. Leonardi: Studies examining the synergy between Dihydrotanshinone and Temozolomide against MGMT+ glioblastoma cells in vitro: Predicting interactions with the blood-brain barrier. Biomed Pharmacother, 109, 386-390 (2019) doi:10.1016/j.biopha.2018.10.069
37. D. P. Radin, R. Purcell and A. S. Lippa: Oncolytic Properties of Ampakines In Vitro. Anticancer Res, 38(1), 265-269 (2018) doi:10.21873/anticanres.12217
38. J. Xu, N. H. Patel, T. Saleh, E. K. Cudjoe, Jr., M. Alotaibi, Y. Wu, S. Lima, A. M. Hawkridge and D. A. Gewirtz: Differential Radiation Sensitivity in p53 Wild-Type and p53-Deficient Tumor Cells Associated with Senescence but not Apoptosis or (Nonprotective) Autophagy. Radiat Res, 190(5), 538-557 (2018) doi:10.1667/RR15099.l
39. M. Tamamori-Adachi, A. Koga, T. Susa, H. Fujii, M. Tsuchiya, H. Okinaga, H. Hisaki, M. Iizuka, S. Kitajima and T. Okazaki: DNA damage response induced by Etoposide promotes steroidogenesis via GADD45A in cultured adrenal cells. Sci Rep, 8(1), 9636 (2018) doi: I 0.1038/s41598-018-27938-5
40. I. 0. Racoma, W. H. Meisen, Q. E. Wang, B. Kaur and A. A. Wani: Thymoquinone inhibits autophagy and induces cathepsin-mediated, caspase-independent cell death in glioblastoma cells. PLoS One, 8(9), e72882 (2013) doi:10.1371/journal.pone.0072882
41. L. Ferrer-Font, L. Villamanan, N. Arias-Ramos, J. Vilardell, M. Plana, M. Ruzzene, L. A. Pinna, E. Itarte, C. Arns and A. P. Candiota: Targeting Protein Kinase CK2: Evaluating CX-4945 Potential for GL261 Glioblastoma Therapy in Immunocompetent Mice. Pharmaceuticals (Basel), 10(1) (2017) doi:10.3390/phl0010024
42. N. B. Roberts, A. Alqazzaz, J. R. Hwang, X. Qi, A. D. Keegan, A. J. Kim, J. A. Winkles and G. F. Woodworth: Oxaliplatin disrupts pathological features of glioma cells and associated macrophages independent of apoptosis induction. J Neurooncol, 140(3), 497-507 (2018) doi:10.1007/s11060-018-2979-1
43. C. P. Couturier, S. Ayyadhury, P. U. Le, J. Nadaf, J. Monlong, G. Riva, R. Allache, S. Baig, X. Yan, M. Bourgey, C. Lee, Y. C. D. Wang, V. Wee Yong, M. C. Guiot, H. Najafabadi, B. Misic, J. Ante!, G. Bourque, J. Ragoussis and K. Petrecca: Single-cell RNA-seq reveals that glioblastoma recapitulates a normal neurodevelopmental hierarchy. Nat Commun, 11(1), 3406 (2020) doi:10.1038/s41467-020-17186-5
44. S. Bao, Q. Wu, R. E. McLendon, Y. Hao, Q. Shi, A. B. Hjelmeland, M. W. Dewhirst, D. D. Bigner and J. N. Rich: Glioma stem cells promote radioresistance by preferential activation of the DNA damage response. Nature, 444(7120), 756-60 (2006) doi:10.1038/nature05236
45. J. Chen, Y. Li, T. S. Yu, R. M. McKay, D. K. Burns, S. G. Kernie and L. F. Parada: A restricted cell population propagates glioblastoma growth after chemotherapy. Nature, 488(7412), 522-6 (2012) doi:10.1038/naturel 1287
46. S. Abbas, S. K. Singh, A. K. Saxena, S. Tiwari, L. K. Sharma and M. Tiwari: Role of autophagy in regulation of glioma stem cells population during therapeutic stress. J Stem Cells Regen Med, 16(2), 80-89 (2020) doi:10.46582/jsrm.1602012
47. M. Buccarelli, M. Marconi, S. Pacioni, I. De Pascalis, Q. G. D'Alessandris, M. Martini, B. Ascione, W. Malorni, L. M. Larocca, R. Pallini, L. Ricci-Vitiani and P. Matarrese: Inhibition of autophagy increases susceptibility of glioblastoma stem cells to temozolomide by igniting ferroptosis. Cell Death Dis, 9(8), 841 (2018) doi:10.1038/s41419-018-0864-7
48. J. Li, Y. Sun, X. Sun, X. Zhao, Y. Ma, Y. Wang and X. Zhang: AEG-1 silencing attenuates M2-polarization of glioma-associated microglia/macrophages and sensitizes glioma cells to temozolomide. Sci Rep, 11(1), 17348 (2021) doi:10.1038/s41598-021-96647-3
49. G. Lee, B. Auffinger, D. Guo, T. Hasan, M. Deheeger, A. L. Tobias, J. Y. Kim, F. Atashi, L. Zhang, M. S. Lesniak, C. D. James and A. U. Ahmed: Dedifferentiation of Glioma Cells to Glioma Stem-like Cells By Therapeutic Stress-induced HIF Signaling in the Recurrent GBM Model. Mal Cancer Ther, 15(12), 3064-3076 (2016) doi:10.1158/1535-7163.MCT-15-0675
50. J. H. Azambuja, E. F. da Silveira, T. R. de Carvalho, P. S. Oliveira, S. Pacheco, C. T. do Couto, F. T. Beira, F. M. Stefanello, R. M. Spanevello and E. Braganhol: Glioma sensitive or chemoresistant to temozolomide differentially modulate macrophage protumor activities. Biochim Biophys Acta Gen Subj, 1861(11 Pt A), 2652-2662 (2017) doi:10.1016/j.bbagen.2017.07.007
51. S. Bastola, M. S. Pavlyukov, D. Yamashita, S. Ghosh, H. Cho, N. Kagaya, Z. Zhang, M. Minata, Y. Lee, H. Sadahiro, S. Yamaguchi, S. Komarova, E. Yang, J. Markert, L. B. Nabors, K. Bhat, J. Lee, Q. Chen, D. K. Crossman, K. Shin-Ya, D. H. Nam and I. Nakano: Glioma-initiating cells at tumor edge gain signals from tumor core cells to promote their malignancy. Nat Commun, 11(1), 4660 (2020) doi:10.1038/s41467-020-18189-y
52. R. B. Puchalski, N. Shah, J. Miller, R. Dalley, S. R. Nomura, J. G. Yoon, K. A. Smith, M. Lankerovich, D. Bertagnolli, K. Bickley, A. F. Boe, K. Brouner, S. Butler, S. Caldejon, M. Chapin, S. Datta, N. Dee, T. Desta, T. Dolbeare, N. Dotson, A. Ebbert, D. Feng, X. Feng, M. Fisher, G. Gee, J. Goldy, L. Gourley, B. W. Gregor, G. Gu, N. Hejazinia, J. Hohmann, P. Hothi, R. Howard, K. Joines, A. Kriedberg, L. Kuan, C. Lau, F. Lee, H. Lee, T. Lemon, F. Long, N. Mastan, E. Mott, C. Murthy, K. Ngo, E. Olson, M. Reding, Z. Riley, D. Rosen, D. Sandman, N. Shapovalova, C. R. Slaughterbeck, A. Sodt, G. Stockdale, A. Szafer, W. Wakeman, P. E. Wohnoutka, S. J. White, D. Marsh, R. C. Rostomily, L. Ng, C. Dang, A. Jones, B. Keogh, H. R. Gittleman, J. S. Barnholtz-Sloan, P. J. Cimino, M. S. Uppin, C. D. Keene, F. R. Farrokhi, J. D. Lathia, M. E. Berens, A. Iavarone, A. Bernard, E. Lein, J. W. Phillips, S. W. Rostad, C. Cobbs, M. J. Hawrylycz and G. D. Foltz: An anatomic transcriptional atlas of human glioblastoma. Science, 360(6389), 660-663 (2018) doi:10.1126/science.aaf2666
53. C. Caruso Bavisotto, G. Alberti, A. M. Vitale, L. Paladino, C. Campanella, F. Rappa, M. Gorska, E. Conway de Macario, F. Cappello, A. J. L. Macario and A. Marino Gammazza: Hsp60 Post-translational Modifications: Functional and Pathological Consequences. Front Mal Biosci, 7, 95 (2020) doi:10.3389/fmolb.2020.00095
54. H. Maes, A. Kuchnio, A. Perie, S. Moens, K. Nys, K. De Bock, A. Quaegebeur, S. Schoors, M. Georgiadou, J. Wouters, S. Vinckier, H. Vankelecom, M. Garmyn, A. C. Vion, F. Radtke, C. Boulanger, H. Gerhardt, E. Dejana, M. Dewerchin, B. Ghesquiere, W. Annaert, P. Agostinis and P. Carmeliet: Tumor vessel normalization by chloroquine independent of autophagy. Cancer Cell, 26(2), 190-206 (2014) doi:10.1016/j.ccr.2014.06.025
55. Z. Li, S. Bao, Q. Wu, H. Wang, C. Eyler, S. Sathornsumetee, Q. Shi, Y. Cao, J. Lathia, R. E. Mclendon, A. B. Hjelmeland and J. N. Rich: Hypoxia-inducible factors regulate tumorigenic capacity of glioma stem cells. Cancer Cell, 15(6), 501-13 (2009) doi:10.1016/j.ccr.2009.03.018
56. B. Yang, F. Wang and G. Zheng: Transmembrane protein TMEMl 19 facilitates the sternness of breast cancer cells by activating Wnt/beta-catenin pathway. Bioengineered, 12(1), 4856-4867 (2021) doi:10.1080/21655979.2021.1960464
57 E. E. Mulkearns-Hubert, L. A. Torre-Healy, D. J. Silver, J. T. Eurich, D. Bayik, E. Serbinowski, M. Hitomi, J. Zhou, B. Przychodzen, R. Zhang, S. A. Sprowls, J. S. Hale, T. J. Alban, A. Berezovsky, B. A. Bell, P. R. Lockman, B. K. Jha and J. D. Lathia: Development of a Cx46 Targeting Strategy for Cancer Stem Cells. Cell Rep, 27(4), 1062-1072 e5 (2019) doi:10.1016/j.celrep.2019.03.079
58. J. Shi, X. Dong, H. Li, H. Wang, Q. Jiang, L. Liu, L. Wang and J. Dong: Nicardipine sensitizes temozolomide by inhibiting autophagy and promoting cell apoptosis in glioma stem cells. Aging (Albany NY), 13(5), 6820-6831 (2021) doi:10.18632/aging.202539
59. E. H. Kim, Y. Jo, S. Sai, M. J. Park, J. Y. Kim, J. S. Kim, Y. J. Lee, J. M. Cho, S. Y. Kwak, J. H. Baek, Y. K. Jeong, J. Y. Song, M. Yoon and S. G. Hwang: Tumor-treating fields induce autophagy by blocking the Akt2/miR29b axis in glioblastoma cells. Oncogene, 38(39), 6630-6646 (2019) doi:10.1038/s41388-019-0882-7
60. A. Shteingauz, Y. Porat, T. Voloshin, R. S. Schneiderman, M. Munster, E. Zeevi, N. Kaynan, K. Gotlib, M. Giladi, E. D. Kirson, U. Weinberg, A. Kinzel and Y. Palti: AMPK-dependent autophagy upregulation serves as a survival mechanism in response to Tumor Treating Fields (TTFields). Cell Death Dis, 9(11), 1074 (2018) doi:10.1038/s41419-018-1085-9
61. Y. Yan, Z. Xu, S. Dai, L. Qian, L. Sun and Z. Gong: Targeting autophagy to sensitive glioma to temozolomide treatment. J Exp Clin Cancer Res, 35, 23 (2016) doi:10.1186/s13046-016-0303-5
62. L. Roy, M. Poirier and D. Fortin: Pl 1.39 Chloroquine as an anti-glioblastoma therapeutic: repurposing of an old drug. Neuro-Oncology, 21(Supplement_3), iii51-iii52 (2019) doi:10.1093/neuonc/noz126.185
63. W. Zhou, Y. Guo, X. Zhang and Z. Jiang: Lys05 induces lysosomal membrane permeabilization and increases radiosensitivity in glioblastoma. J Cell Biochem, 121(2), 2027-2037 (2020) doi:10.1002/jcb.29437
64. K. A. Jacobs, G. Andre-Gregoire, C. Maghe, A. Thys, Y. Li, E. Harford-Wright, K. Trillet, T. Douanne, C. Alves Nicolau, J. S. Frenel, N. Bidere and J. Gavard: Paracaspase MALTl regulates glioma cell survival by controlling endo-lysosome homeostasis. EMBO J, 39(1), el02030 (2020) doi:10.15252/embj.2019102030
65. V. Le Joncour, P. Filppu, M. Hyvonen, M. Holopainen, S. P. Turunen, H. Sihto, I. Burghardt, H. Joensuu, 0. Tynninen, J. Jaaskelainen, M. Weller, K. Lehti, R. Kakela and P. Laakkonen: Vulnerability of invasive glioblastoma cells to lysosomal membrane destabilization. EMBO Mo! Med, 11(6) (2019) doi:10.15252/emmm.201809034
66. P. Maycotte, C. M. Gearheart, R. Barnard, S. Aryal, J. M. Mulcahy Levy, S. P. Fosmire, R. J. Hansen, M. J. Morgan, C. C. Porter, D. L. Gustafson and A. Thorburn: STAT3-mediated autophagy dependence identifies subtypes of breast cancer where autophagy inhibition can be efficacious. Cancer Res, 74(9), 2579-90 (2014) doi:10.1158/0008-5472.CAN-13-3470
67. 0. A. Guryanova, Q. Wu, L. Cheng, J. D. Lathia, Z. Huang, J. Yang, J. MacSwords, C. E. Eyler, R. E. McLendon, J. M. Heddleston, W. Shou, D. Hambardzumyan, J. Lee, A. B. Hjelmeland, A. E. Sloan, M. Brede!, G. R. Stark, J. N. Rich and S. Bao: Nonreceptor tyrosine kinase BMX maintains self-renewal and tumorigenic potential of glioblastoma stem cells by activating STAT3. Cancer Cell, 19(4), 498-511 (2011) doi:10.1016/j.ccr.2011.03.004
68. S. Mehta, E. Huillard, S. Kesari, C. L. Maire, D. Golebiowski, E. P. Harrington, J. A. Alberta, M. F. Kane, M. Theisen, K. L. Ligon, D. H. Rowitch and C. D. Stiles: The central nervous system-restricted transcription factor Olig2 opposes p53 responses to genotoxic damage in neural progenitors and malignant glioma. Cancer Cell, 19(3), 359-71 (2011) doi:10.1016/j.ccr.2011.01.035
69. H. Liu, W. Weng, R. Guo, J. Zhou, J. Xue, S. Zhong, J. Cheng, M. X. Zhu, S. J. Pan and Y. Li: Olig2 SUMOylation protects against genotoxic damage response by antagonizing p53 gene targeting. Cell Death Differ, 27(11), 3146-3161 (2020) doi:10.1038/s41418-020-0569-1
70. R. P. Dings, M. Loren, H. Heun, E. McNiel, A. W. Griffioen, K. H. Mayo and R. J. Griffin: Scheduling of radiation with angiogenesis inhibitors anginex and Avastin improves therapeutic outcome via vessel normalization. Clin Cancer Res, 13(11), 3395-402 (2007) doi:10.1158/1078-0432.CCR-06-244l
71. J. Sheehan, C. P. Cifarelli, K. Dassoulas, C. Olson, J. Rainey and S. Han: Trans-sodium crocetinate enhancing survival and glioma response on magnetic resonance imaging to radiation and temozolomide. J Neurosurg, 113(2), 234-9 (2010) doi:10.3171/2009.11.JNS091314
72. M. Z. Noman, B. Janji, B. Kaminska, K. Van Moer, S. Pierson, P. Przanowski, S. Buart, G. Berchem, P. Romero, F. Mami-Chouaib and S. Chouaib: Blocking hypoxia-induced autophagy in tumors restores cytotoxic I-cell activity and promotes regression. Cancer Res, 71(18), 5976-86 (2011) doi:10.1158/0008-5472.CAN-11-1094
73. D. P. Radin and S. E. Tsirka: Interactions between Tumor Cells, Neurons, and Microglia in the Glioma Microenvironment. Int J Mo! Sci, 21(22) (2020) doi:10.3390/ijms21228476
74. J. Li, M. M. Kaneda, J. Ma, M. Li, R. M. Shepard, K. Patel, T. Koga, A. Sarver, F. Furnari, B. Xu, S. Dhawan, J. Ning, H. Zhu, A. Wu, G. You, T. Jiang, A. S. Venteicher, J. N. Rich, C. K. Glass, J. A. Varner and C. C. Chen: PI3Kgamma inhibition suppresses microglia/TAM accumulation in glioblastoma microenvironment to promote exceptional temozolomide response. Prac Natl Acad Sci USA, 118(16) (2021) doi:10.1073/pnas.2009290118
75. H. Y. Chuang, Y. K. Su, H. W. Liu, C. H. Chen, S. C. Chiu, D. Y. Cho, S. Z. Lin, Y. S. Chen and C. M. Lin: Preclinical Evidence of STAT3 Inhibitor Pacritinib Overcoming Temozolomide Resistance via Downregulating miR-21-Enriched Exosomes from M2 Glioblastoma-Associated Macrophages. J Clin Med, 8(7) (2019) doi:10.3390/jcm8070959
76. L. Akkari, R. L. Bowman, J. Tessier, F. Klemm, S. M. Handgraaf, M. de Groot, D. F. Quail, L. Tillard, J. Gadiot, J. T. Huse, D. Brandsma, J. Westerga, C. Watts and J. A. Joyce: Dynamic changes in glioma macrophage populations after radiotherapy reveal CSF-1R inhibition as a strategy to overcome resistance. Sci Transl Med, 12(552) (2020) doi:10.1126/scitranslmed.aaw7843
77. G. Sharma, R. Ojha, E. Noguera-Ortega, V. W. Rebecca, J. Attanasio, S. Liu, S. Piao, J. J. Lee, M. C. Nicastri, S. L. Harper, A. Ronghe, V. Jain, J. D. Winkler, D. W. Speicher, J. Mastio, P. A. Gimotty, X. Xu, E. J. Wherry, D. I. Gabrilovich and R. K. Amaravadi: PPTl inhibition enhances the antitumor activity of anti-PD-I antibody in melanoma. JC! Insight, 5(17) (2020) doi:10.1172/jci.insight.133225
78. V. W. Rebecca, M. C. Nicastri, C. Fennelly, C. I. Chude, J. S. Barber-Rotenberg, A. Ronghe, Q. McAfee, N. P. McLaughlin, G. Zhang, A. R. Goldman, R. Ojha, S. Piao, E. Noguera-Ortega, A. Martorella, G. M. Alicea, J. J. Lee, L. M. Schuchter, X. Xu, M. Herlyn, R. Marmorstein, P. A. Gimotty, D. W. Speicher, J. D. Winkler and R. K. Amaravadi: PPTl Promotes Tumor Growth and Is the Molecular Target of Chloroquine Derivatives in Cancer. Cancer Discav, 9(2), 220-229 (2019) doi:10.1158/2159-8290.CD-18-0706
79. M. C. Nicastri, V. W. Rebecca, R. K. Amaravadi and J. D. Winkler: Dimeric quinacrines as chemical tools to identify PPTl, a new regulator of autophagy in cancer cells. Mal Cell Oneal, 5(1), e1395504 (2018) doi:10.1080/23723556.2017.1395504

What is claimed:

1. A method of treating or preventing a central nervous system (CNS) cancer in a mammalian subject, comprising:
Administering to the subject a lucanthone compound or composition in an amount, dosage or regimen effective to reduce one or more adverse clinical symptom(s) of the CNS cancer, and/or to extend average survival among treated subjects.

2. The method of claim 1, wherein the CNS cancer is a glioblastoma.

3. The method of claim 1, wherein the lucanthone compound or composition effectively reduces an incidence or severity of chemoresistance and/or disease recurrence associated with conventional chemotherapy treatment.

4. The method of claim 1, wherein the CNS cancer is glioblastoma, and wherein the lucanthone compound or composition effectively reduces an incidence or severity of chemoresistance and disease recurrence associated with conventional glioblastoma treatment employing a temozolomide compound or composition.

5. The method of claim 1, wherein the lucanthone compound or composition effectively suppresses growth of stem cells corresponding to a targeted CNS cancer.

6. The method of claim 1, wherein the CNS cancer is glioblastoma and wherein the lucanthone compound or composition effectively inhibits or eliminates TMEMl 19+ glioma cells in tumors.

7. The method of claim 1, wherein the CNS cancer is glioblastoma and wherein the lucanthone compound or composition effectively suppresses growth of glioblastoma stem cells

8. The method of claim 1, wherein the CNS cancer is glioblastoma and wherein the lucanthone compound or composition effectively inhibits or eliminates Olig2+ glioma cells in tumors or in circulation within treated subjects.

9. The method of claim 1, wherein the CNS cancer is glioblastoma and wherein the lucanthone compound or composition effectively normalizes tumor vasculature in treated versus control subjects.

10. The method of claim 1, wherein the CNS cancer is glioblastoma and wherein the lucanthone compound or composition effectively reduces tumor hypoxia in treated versus control subjects.

11. A method of treating or preventing a central nervous system (CNS) cancer in a mammalian subject, comprising:
Administering to the subject a lucanthone compound or composition coordinately with a temozolomide compound or composition, in respective amounts, dosages or regimens effective to coordinately or complementarily reduce one or more adverse clinical symptom(s) of the CNS cancer, and/or to extend average survival among treated subjects.

12. The method of claim 11, wherein the CNS cancer is a glioblastoma.

13. The method of claim 11, wherein the lucanthone compound or composition and temozolomide compound or composition coordinately or complementarily reduce an incidence or severity of chemoresistance and disease recurrence associated with conventional chemotherapy treatment.

14. The method of claim 11, wherein the CNS cancer is glioblastoma, and wherein the lucanthone compound or composition and temozolomide compound or composition coordinately or complementarily reduce an incidence or severity of chemoresistance and disease recurrence associated with conventional glioblastoma treatment employing the temozolomide compound or composition alone.

15. The method of claim 11, wherein the lucanthone compound or composition and temozolomide compound or composition coordinately or complementarily suppress growth of stem cells corresponding to a targeted CNS cancer.

16. The method of claim 11, wherein the CNS cancer is glioblastoma and wherein the lucanthone compound or composition effectively inhibits or eliminates TMEMl 19+ glioma cells in tumors.

17. The method of claim 11, wherein the CNS cancer is glioblastoma and wherein the lucanthone compound or composition and temozolomide compound or composition coordinately or complementarily suppress growth of glioblastoma stem cells

18. The method of claim 11, wherein the CNS cancer is glioblastoma and wherein the lucanthone compound or composition and temozolomide compound or composition coordinately or complementarily inhibit or eliminate Olig2+ glioma cells in tumors.

19. The method of claim 11, wherein the CNS cancer is glioblastoma and wherein the lucanthone compound or composition and temozolomide compound or composition coordinately or complementarily normalize tumor vasculature in treated versus control subjects.

20. The method of claim 11, wherein the CNS cancer is glioblastoma and wherein the lucanthone compound or composition and temozolomide compound or composition coordinately or complementarily reduce tumor hypoxia in treated versus control subjects.

21. A pharmaceutical composition or kit for treating or preventing a central nervous system (CNS) cancer in a mammalian subject, comprising:
A lucanthone compound or composition co-formulated or packaged for coordinate clinical use with a temozolomide compound, in respective amounts or dosages to effectively reduce one or more adverse clinical symptom(s) of the CNS cancer, and/or to extend average survival among treated subjects.

22. The pharmaceutical composition or kit of claim 20, wherein the CNS cancer is a glioblastoma.

23. A method of treating or preventing a central nervous system (CNS) cancer in a mammalian subject, comprising:
Administering to the subject a lucanthone compound or composition coordinately with ionizing radiation, in respective amounts, dosages or regimens effective to reduce one or more adverse clinical symptom(s) of the CNS cancer, and/or to extend average survival among treated subjects.

24. The method of claim 23, wherein the CNS cancer is a glioblastoma.

25. The method of claim 23, wherein the lucanthone compound or composition and radiation coordinately or complementarily reduce an incidence or severity of chemoresistance and disease recurrence associated with conventional chemotherapy or radiation treatment.

26. The method of claim 23, wherein the CNS cancer is glioblastoma, and wherein the lucanthone compound or composition and radiation coordinately or complementarily reduce an incidence or severity of radiation resistance or chemoresistance and disease recurrence associated with conventional glioblastoma treatment employing the radiation alone.

27. The method of claim 23, wherein the lucanthone compound or composition and radiation coordinately or complementarily suppress growth of stem cells corresponding to a targeted CNS cancer.

28. The method of claim 23, wherein the CNS cancer is glioblastoma and wherein the lucanthone compound or composition and radiation coordinately or complementarily suppress growth of glioblastoma stem cells

29. The method of claim 23, wherein the CNS cancer is glioblastoma and wherein the lucanthone compound or composition and radiation coordinately or complementarily inhibit or eliminate Olig2+ glioma cells in tumors.

30. The method of claim 23, wherein the CNS cancer is glioblastoma and wherein the lucanthone compound or composition and radiation coordinately or complementarily normalize tumor vasculature in treated versus control subjects.

31. The method of claim 23, wherein the CNS cancer is glioblastoma and wherein the lucanthone compound or composition and radiation coordinately or complementarily reduce tumor hypoxia in treated versus control subjects.

32. The method of claim 23, wherein the CNS cancer is glioblastoma and wherein the lucanthone compound or composition effectively inhibits or eliminates TMEMl 19+ glioma cells in tumors.
